(19) United States
(12) Patent Application Publication
(10) Pub. No.: US 2011/0223675 A1
(43) Pub. Date: Sep. 15, 2011

(54) DRUG RELEASE MEANS FROM LIPOSOMES AND METHOD FOR EVALUATING RELEASABILITY

(75) Inventors: Koji Nakamura, Kanagawa (JP); Keisuke Yoshino, Kanagawa (JP); Masayo Kondo, Kanagawa (JP); Keiko Yamashita, Kanagawa (JP)

(73) Assignee: TERUMO KABUSHIKI KAISHA, Shibuya-ku (JP)

(21) Appl. No.: 13/130,513

(22) PCT Filed: Nov. 20, 2009
(86) PCT No.: PCT/JP2009/069710

§ 371 Date: May 20, 2011

(30) Foreign Application Priority Data
Nov. 20, 2008 (JP) 2008297400

(51) Int. Cl. G01N 33/15

(52) U.S. Cl. 436/71

(57) ABSTRACT

Drug release means from liposomes and a method for evaluating drug releasability of liposome preparations, which are useful for the quality control of a liposome preparation, are simple, accurate and excellent in reproducibility and are able to achieve in vivo/in vitro correlation (IVIVC), are provided. The drug release means from liposomes by causing the shift of chemical equilibrium in the inside of the liposome and the method for evaluating drug releasability by quantitatively determining the drug released to the outside of the liposome.

### TECHNICAL FIELD

This invention relates to drug release means from liposomes and also to a method for evaluating drug releasability of liposome preparations making use of the release means.

### BACKGROUND ART

### <Liposomes Serving as a DDS Medium>

Liposomes are closed vesicles, which have been discovered by Bangham et al., in the 1960s and are formed of phospholipids, and have been initially being studied as a biological membrane model. Thereafter, investigations of applications to DDS making use of the inherent characteristic of liposomes have been in progress and thus, the liposomes have become well known as one of DDS mediums at present.

### <Method of Preparing Liposomes>

For a method of preparing liposomes, there is generally well known a hydration method (Bangham method). Although depending on the difference in several operations, this is called an ultrasonic treatment method and also an extrusion method whose fundamental operations are the same and is the simplest liposome preparation method.

More particularly, liposomes can be prepared by preparing a phospholipid in the form of a thin membrane, adding an aqueous solvent thereto to cause hydration/swelling, and subjecting to ultrasonic treatment or extrusion.

Where a lipophilic drug is entrapped, the drug is dissolved simultaneously with the stage where the phospholipid membrane is prepared, thereby permitting the drug to be incorporated into the phospholipid membrane.

On the other hand, where a water-soluble drug is entrapped, the drug is dissolved in an aqueous solvent used for hydration/swelling and is entrapped in an aqueous phase (hereinafter referred to as “inner aqueous phase”) inside the liposomes by ultrasonic treatment or extrusion.

As stated above, although these preparation methods are the simplest method, it has been accepted that a problem is involved in that the entrapping efficiency of the drug is low. More particularly, with a lipophilic drug, it is incorporated into the lipid membrane, not into the inner aqueous phase, thus enabling it impossible to entrap the drug in amounts not less than the moles of the lipid. With a water-soluble drug, although the drug is entrapped in the inner aqueous phase, the inner entrapment is feasible only at a ratio of the inner aqueous phase to an outer aqueous phase (which means an aqueous phase of an outer portion of liposome herein and whenever it appears hereinafter), so that there is a limit wherein several tens of percent of the total drug is entrapped in the inner aqueous phase. This method is called a passive loading method.

As a method of solving the problem of the drug entrapping efficiency, there is a remote loading method (Patent Documents 1, 2). According to the remote loading method, a drug can be stably introduced at a high entrapping efficiency.

### <Remote Loading Method>

One example of the remote loading method is illustrated below.

For an outer aqueous phase of liposome, there is used a buffer solution whose pH is properly adjusted. This outer aqueous phase makes use of an ammonium ion-free medium (e.g. NaCl or a sugar) and the inner aqueous phase and outer aqueous phase of liposomes are, respectively, controlled in osmotic pressure within ranges where the liposomes are not broken down owing to the difference in osmotic pressure therebetween.

The ammonium ion in the liposomes is in equilibrium with ammonia and a proton. Non-protonated ammonia freely permeates the lipid bilayer membrane of liposomes and can be migrated to outside of the liposomes. Hence, there arises a phenomenon that the equilibrium is continuously shifted inside the liposome.

This remote loading method is an entrapping method, which is applicable to commonly-used drugs that are able to exist in a charged form in case where dissolved in an appropriate aqueous medium and is thus limited to such drugs. Typically, an ion gradient is formed between the inner and outer sides of liposomes, so that a drug permeates the liposome membrane according to the formed gradient thereby permitting the drug to be entrapped in the liposomes. In so far as there are used drugs of the type to which such an introduction method is applicable, they are internally entrapped at an entrapping efficiency close to 100% (Patent Documents 3 to 5, Non-patent Document 1).

### <Drug Release and Drug Releasability Testing Method>

The liposomes, in which a drug is entrapped according to the remote loading method, are stored as a preparation in a container such as a vial, the ion gradient upon the entrapping is kept, and the entrapped drug is held in the inner aqueous phase of the liposomes with no release occurring.

However, it is known that after administration into a living body, there is some possibility of causing the release ascribed to a factor of some sort and the release profile differs depending on the type of drug to be entrapped.

For instance, with the case of liposomes entrapping doxorubicin hydrochloride therein, no release occurs after their administration into a living body and the disposition of the liposomes and the disposition of the entrapped drug are substantially the same. However, most of drugs capable of being entrapped by the remote loading method are preparations of the type wherein release occurs rapidly after administration into a living body, and these are ranked as a slow-release preparation (Patent Documents 4, 6, 7 and Non-patent Document 2).

In view of the production and distribution of such slow-release preparations, the drug release characteristics from the liposome preparations are required, as part of quality assurance and quality control, to be invariably within a certain standard range and their confirmation is needed.

In general, the release of a drug from liposomes is influenced by an external environment and physical and chemical characteristics of a liposome preparation. Accordingly, the method of evaluating drug release characteristics of a liposome preparation should preferably be one wherein the drug release characteristics based on an external environment and the physical and chemical characteristics of a liposome membrane can be evaluated simultaneously.

For a method of evaluating physical and chemical characteristics of a liposome membrane, there are methods including differential scanning calorimetry, an electron spin resonance (ESR) method making use of electron resonance, a nucleic magnetic resonance (NMR) method, a fluorescence method utilizing a spectral change or a change in degree of deflection of a fluorescence probe or the like (hereinafter referred to as “related-art method 1”).

On the other hand, it is considered to use an ordinarily known release testing method established for oral administration preparations as a liposome preparation release testing method. This is to evaluate the drug releasability ascribed to preparation disintegration in a buffer solution assumed as a gastrointestinal tract fluid (hereinafter referred to as “related-art method 2”).

The drug release characteristics from liposomes can be evaluated by an in vivo test in the practice of a laboratory level and by an in vitro test making use of components of a living body or living body-derived components such as blood serum, blood plasma and the like (hereinafter referred to as “related-art method 3”).

There is further disclosed, as a method differing in standpoint from the above methods, a simple method of evaluating drug release characteristics of a liposome preparation wherein a state within liposomes is mimicked in a test tube prior to liposomization and is observed to evaluate the drug retentivity of the liposomes (Patent Document 8, hereinafter referred to as “related-art method 4”).

### PRIOR ART DOCUMENT

### Patent Document

Patent Document 1: U.S. Pat. No. 5,192,549

Patent Document 2: U.S. Pat. No. 5,316,771

Patent Document 3: U.S. Pat. No. 5,077,056

Patent Document 4: U.S. Pat. No. 5,837,282

Patent Document 5: U.S. Published Application No. 2002/0110586

Patent Document 6: PCT Patent Publication Pamphlet No. WO01/00173

Patent Document 7: U.S. Published Application No. 2007/0231379

Patent Document 8: PCT Patent Publication Pamphlet No. WO03/032947

### Non-Patent Document

Non-patent Document 1: Written by Gregoriadis, G. “Liposome Technology,” 2nd edition, Vol. 1, Liposome Preparation and Related Technologies, U.S.A., CRC press, Dec. 13, 1992

Non-patent Document 2: Zamboni, W C et al., “Plasma, Tumor, and Tissue Disposition of STEALTH liposomal CKD-602 (S-CKD602) and Nonliposomal CKD-602 in Mice Bearing A375 Human Melanoma Xenografts,” Clinical Cancer Research, 2007, Vol. 13, No. 23, pp. 7217-7223

### SUMMARY OF INVENTION

### Technical Problem

### <Problems of Related-Art Drug Releasability Testing Methods>

With respect to the related-art methods 1, most of them are not suited for use as a drug releasability testing method of a liposome preparation because they need certain types of additives, the introduction of a specific type of large-scaled equipment, or a complicated measuring procedure, not permitting operations to be carried out simply.

In the related-art method 2, not only administration route of a liposome preparation relies mainly on parenteral administration, but also a drug is stably held in the liposomes (especially, in respect of liposome preparations prepared according to the remote loading method). Hence, mere dispersion in a buffer solution does not cause drug release. Additionally, because no drug release occurs by disintegration of liposomes, diversion as a drug releasability testing method of liposome preparations is not proper.

With respect to the related-art method 3, when the method is carried out at an industrial level, there are involved problems on accuracy, reproducibility and the like, which are ascribed to the individual differences of animals, lot-to-lot differences of living body components and storage stability of living body components. Thus, it is not useful as a drug releasability testing method of liposome preparations.

The related-art method 4 is able to relatively compare a number of drugs with one another and is thus useful from the standpoint that drugs capable of being entrapped in liposomes are subjected to screening. Nevertheless, this method is one wherein release is forecasted absolutely prior to liposomization, and the actual drug release characteristics of liposome preparations cannot be directly measured. Accordingly, it is not proper to utilize this method as a drug releasability testing method of liposome preparations.

As stated hereinabove, applications of the related-art methods have made it difficult to accurately, simply reproducibly test and evaluate drug release characteristics of liposome slow-release preparations.

### Objects of the Invention

The invention is to solve the problems involved in the related-art drug releasability testing methods and has for its object the provision of drug release means from liposomes and a method for evaluating drug releasability of liposome preparations, which are able to measure drug releasability of a liposome preparation entrapping a drug in an in vitro system without use of a human body, an experimental animal, cultured cells, or a living body or living body-derived substances such as blood serum, blood plasma and the like, are simple, correct and excellent in reproducibility and are able to achieve in vivo/in vitro correlation (IVIVC).

### Technical Solution

In order to achieve the above object, the present inventors made intensive studies and, as a result, found that when liposomes entrapping a drug in an inner aqueous phase are permitted to be present in a solution, to which a shift reagent is added and a concentration of the drug in the solution is measured, drug releasability of a liposome preparation can be assessed, thereby arriving at completion of the invention. In one embodiment of the invention, a deprotonation reagent or protonation reagent is used as the shift reagent.

In the practice of the invention, the “shift reagent” means a reagent wherein when used, it is able to create an environment likely to release a drug from the inside of liposomes by forming an ion gradient, which is opposite to an ion gradient formed at the time of drug entrapping, between the inner aqueous phase and the outer aqueous phase of liposomes entrapping a drug therein, and weakening the ion gradient formed to hold the drug, thereby causing the shift of chemical equilibrium of the inner aqueous phase to weaken the retention of the drug held as dissolved in the inner aqueous phase. In short, it is considered that the action of the shift reagent is to weaken the ion gradient, which is formed between the inner aqueous phase and the outer aqueous phase of liposomes so as to retain a drug.

In the invention, the “deprotonating reagent” or “protonating reagent” serving as an embodiment of the “shift reagent” has the following features:

(1) It is permeable through a phospholipid membrane of liposomes in a non-ionized state; and

(2) A deprotonating reagent serves as a Brønsted base and deprotonates a drug in the inner aqueous phase of liposomes and a protonating reagent serves as a Brønsted acid and protonates a drug.

In the practice of the invention, the term “shift reagent” may be used, in some case, to mean the term “substance” that permits the “deprotonating reagent” or “protonating reagent” to be formed in solutions. For instance, where ammonium acetate is used as a shift reagent, ammonia is formed in a solution as a deprotonating reagent.

It is considered that the function and effect of the deprotonating reagent or protonating reagent in the invention can be illustrated in the following way. More particularly, it is contemplated that the deprotonating reagent or protonating reagent permeates the phospholipid membrane of liposomes and moves from the outer aqueous phase to the inner aqueous phase and acts as a Brønsted base or Brønsted acid in the inner aqueous phase. The shift of the chemical equilibrium related to a drug held as dissolved in the inner aqueous phase of the liposomes is caused according to Le Chatelier's principle, so that drug retentivity in the inner aqueous phase lowers, with the result that the drug is moved from the inner aqueous phase to the outer aqueous phase.

The present invention can evaluate the drug releasability of liposomal preparation by measuring the concentration of the drug released into a solution from drug entrapped into liposomes after adding the deprotonated and protonated reagent into the solution and make the entrapped drug released into the solution, and stopped the release if necessary.

It should be noted that the deprotonating reagent or protonating reagent may be contained in the solution by adding a shift reagent to the outer aqueous phase solution of liposomes.

It will also be noted that the release of the drug from the inner aqueous phase to the outer aqueous phase of liposomes may be started by heating for a given time.

Further, it will be noted that the release of the drug from the inner aqueous phase to the outer aqueous phase of liposomes may be stopped by natural cooling caused by stopping the heating, or by stopping the heating and further by forced cooling such as ice cooling.

In the invention, liposomes are preferably ones wherein a drug is entrapped according to the remote loading method.

In the invention, the drug is preferably made of an amphiphatic compound, more preferably made of an amphiphatic amphoteric compound, an amphiphatic, weakly basic compound or an amphiphatic, weakly acidic compound, and much more preferably made of an amphiphatic, weakly basic compound.

In the invention, a solution such as an outer aqueous phase solution or the like, to which a shift reagent has been added, is preferably one wherein no shift reagent is contained prior to the addition of a shift reagent. In the practice of the invention, a solution such as an outer aqueous phase solution or the like, to which a shift reagent has been added, is preferably one wherein there is contained, as an embodiment of the shift reagent, neither a deprotonating reagent and a conjugated acid thereof serving as a Brønsted base nor a protonating reagent and a conjugated base thereof serving as a Brønsted acid.

In the invention, a solution such as an outer aqueous phase solution or the like, to which a shift reagent has been added, is preferably free of a living body-derived component such as blood serum or blood plasma.

In the invention, a solution used as a solution, to which a shift reagent has been added, includes water, a physiological saline solution, Ringer's solution or a buffer solution, of which the use of the buffer solution is preferred.

The pH of the buffer solution is preferably determined while taking into account the stability of a drug entrapped in liposomes and the constituent composed of a liposome membrane such as a phospholipid, and a general pH of 5 to 9 is preferred.

One embodiment of the invention may be illustrated in the following way.

Liposomes entrapping a drug (symbolized as “A” herein) therein are permitted to be present in a solution containing a deprotonating reagent (symbolized as “B” herein) and the deprotonating reagent is moved from an outer aqueous phase to an inner aqueous phase after permeation through a liposome lipid membrane in a non-ionized state.

The deprotonating reagent (B) accepts a hydrogen ion (proton) from the cationized drug (HA⁺) and is protonated (HB⁺), and the drug (HA⁺) donates the hydrogen ion (proton) to the deprotonizing reagent and is thus deprotonated (A).

Accordingly, the chemical equilibrium of the following formula is established in the inner aqueous phase with respect to the drug and deprotonating reagent.

[Chemical Formula 1]

HA⁺+B [symbol] A+HB⁺ (1)

In the formula,

A: drug (non-ionized state);

HA⁺: drug (cationized state);

B: deprotonating reagent (non-ionized state);

HB⁺: deprotonating reagent (cationized state); and

H⁺: hydrogen ion (proton).

When a molar concentration [B] of the deprotonating reagent (non-ionized state) at the left-hand side becomes large, the chemical equilibrium of the above formula (1) is shifted to the right-hand side according to Le Chatelier's principle. Eventually, the molar concentration [A] of the drug (non-ionized state) becomes so large that the drug moves to the outer aqueous phase by permeation through the liposome lipid membrane.

In the above regard, the deprotonating reagent is sufficient to be present when a shift reagent is permitted to be present in an aqueous solvent. For instance, where a deprotonating reagent used is ammonia (NH₃), a shift reagent is one sufficient to form ammonia in an aqueous solvent and may be ammonia per se or an ammonium salt such as ammonium acetate (CH₃COONH₄) or the like.

Where a drug used is an amphiphatic, weakly basic compound having an amino group and/or an imino group, a preferred deprotonating reagent includes ammonia or a low molecular weight amine having a molecular weight of not larger than 500, of which ammonia is more preferred.

Another embodiment of the invention is illustrated in the following way.

Liposomes entrapping a drug (symbolized herein as “HC”) are permitted to be present in a solution containing a protonating reagent (symbolized herein as “HD”), and the protonating reagent is moved from the outer aqueous phase to the inner aqueous phase after permeation through the liposome lipid membrane in non-ionized state.

The protonating reagent (HD) donates hydrogen ion (proton) to the drug (C⁻) existing in anionized state and is deprotonated (D⁻), whereas the drug (C⁻) accepts the hydrogen ion (proton) from the protonating reagent and is thus protonated (HC).

Accordingly, the chemical equilibrium of the following formula related to the drug and the protonating reagent is established in the inner aqueous phase.

[Chemical Formula 2]

C⁻+HD [symbol] HC+D⁻ (2)

In the formula,

HC: drug (non-ionized state);

C⁻: drug (anionized state);

HD: protonating reagent (non-ionized state);

D−: protonating reagent (anionized state); and

H⁺: hydrogen ion (proton).

When a molar concentration [HD] of the protonating reagent (non-ionized state) at the left-hand side becomes large, the chemical equilibrium of the above formula (2) is shifted to the right-hand side according to Le Chatelier's principle. As a consequence, the molar concentration [HC] of the drug (non-ionized state) becomes so large that the drug moves to the outer aqueous phase by permeation through the liposome lipid membrane.

In the above regard, the protonating reagent is sufficient to be present when a shift reagent is permitted to be present in an aqueous solvent. For instance, where a protonating reagent used is citric acid, a shift reagent is one sufficient to form citric acid in an aqueous solvent and may be citric acid per se or a citric salt such as sodium citrate or the like.

More particularly, the invention is directed to the followings.

[1] A method for evaluating drug releasability of a liposome preparation, wherein liposomes entrapping a drug therein are permitted to be present in a solution, to which a shift reagent has been added, and a concentration of the drug in the solution is measured.

[2] The method recited in [1], wherein a chemical equilibrium is caused to be shifted in an inner aqueous phase of the liposomes, so that the drug is released to an outer aqueous phase of the liposomes.

[3] The method recited in [1] or [2], wherein the liposomes entrapping the drug therein are made of liposomes entrapping the drug according to a remote loading method.

[4] The method recited in any of [1] to [3], wherein the shift reagent permeates a lipid membrane of the liposomes in a non-ionized state, moves from the outer aqueous phase to the inner aqueous phase and is ionized to non-ionize the drug retained in the inner aqueous phase.

[5] The method recited in any of [1] to [4], wherein the solution is made of a buffer solution.

[6] The method recited in any of [1] to [5], wherein the shift reagent is made of a deprotonating reagent or a protonating reagent.

[7] A method for evaluating drug releasability of a liposome preparation including the following steps of:

(1) preparing a solution, to which a shift reagent has been added;

(2) mixing liposomes entrapping a drug therein with the solution (a suspension obtained in this step is hereinafter referred to as “solution A”);

(3) starting release of the drug into the solution;

(4) separating the liposomes from the solution A (a solution obtained in this step and containing a released drug is hereinafter referred to as “solution B”); and

(5) measuring a concentration of the drug contained in the solution B.

[8] The method recited in [7], wherein in the,step (3), the solution A is heated for a given time at a given temperature.

[9] The method recited in [8], wherein the given temperature is between 30 to 40° C.

[10] The method recited in [8] or [9], wherein the given time is between 1 to 180 minutes.

[11] The method recited in any of [7] to [10], further including the following step, between the steps [3] and [4], of:

(3-2) stopping the release of the drug into the solution.

[12] The method recited in [11], wherein in the step (3-2), the solution A is allowed to cool.

[13] The method recited in [11], wherein in the step (3-2), the solution is cooled.

[14] The method recited in [11], wherein in the step (3-2), a stop solution is added to the solution A.

[15] The method recited in [14], wherein the stop solution is free of the shift reagent.

[16] The method recited in [14] or [15], wherein the stop solution acts to reduce a concentration of the shift reagent in a mixed system of the solution A and the stop solution.

[17] The method recited in [14] or [15], wherein the stop solution lowers a solution temperature in the mixed system of the solution A and the stop solution.

[18] The method recited in any of [14] to [17], wherein the stop solution is a solution having a pH in the range of 1.0 to 5.0.

[19] The method recited in any of [7] to [18], wherein the solution is made of a buffer solution.

[20] The method recited in any of [7] to [19], wherein the shift reagent is made of a deprotonating reagent or protonating reagent.

[21] A drug release method from liposomes, wherein liposomes entrapping a drug therein are permitted to be present in a solution, to which a shift reagent has been added.

[22] The drug release means recited in [21], wherein a shift of a chemical equilibrium is caused in an inner aqueous phase of the liposomes and the drug is released from the inner aqueous phase to an outer aqueous phase of the liposomes.

[23] The method recited in [21] or [22], wherein the liposomes entrapping the drug therein are those liposomes entrapping the drug according to a remote loading method.

[24] The method recited in any of [21] to [23], wherein the solution is made of a buffer solution.

[25] The method recited in any of [21] to [24], wherein the shift reagent is made of a deprotonating reagent or protonating reagent.

[26] The method recited in any of [21] to [25], wherein the deprotonating reagent or protonating reagent permeates a lipid membrane of the liposomes in non-oxidized state, moves from the outer aqueous phase to the inner aqueous phase, and is ionized to non-ionize the drug retained in the inner aqueous phase.

[27] The method recited in any of [1] to [26], wherein the shift reagent is made of at least one selected from the group consisting of ammonia and an amino compound.

[28] The method recited in any of [1] to [26], wherein the shift reagent is made of ammonia.

[29] The method recited in [26], wherein the amino compound is made of an amino compound having a molecular weight of not larger than 500.

[30] The method recited in [29], wherein the amino compound is made of at least one selected from the group consisting of methanolamine, ethanolamine, ethylenediamine and triethylamine.

[31] The method recited in any of [1] to [30], wherein the solution, to which the shift reagent has been added, has a pH in the range of 5.5 to 7.5.

[32] The method recited in any of [1] to [31], wherein the solution, to which the shift reagent has been added, has an osmotic pressure in the range of 20 to 400 mOsm.

[33] The method recited in any of [1] to [32], wherein in the mixed system of the solution, to which the shift reagent has been added, and the liposomes, a concentration of the shift reagent is in the range of 1 to 150 mM.

[34] The method recited in any of [1] to [33], wherein the drug is made of an amphiphatic compound.

[35] The method recited in any of [1] to [33], wherein the drug is made of an amphiphatic, weakly basic compound.

[36] The method recited in [35], wherein the amphiphatic, weakly basic compound is made of at least one selected from epirubicin, daunorubicin, idarubicin, mitxanthrone, carcinomycin, N-acetyladriamycin, rubidazone, 5-imidodaunomycin, N-acetyldaunomycin, all anthracylin products, daunorylin, topotecan, 9-aminocamptotechin, 10,11-methylenedioxycamptotechin, 9-nitrocamptotechin, TAS103, 7-(4-methyl-piperadino-methylene)-10,11-ethylenedioxy-20(S)-camptotechin, 7-(2-isopropylamino)ethyl-20(S)-camptotechin, CKD-602, UCN-01, propranolol, pentamidine, dibucaine, bupivacaine, tetracaine, procaine, chlorpromazine, vinblastine, vincristine, vinorelbine, vindesine, mitomycin C, pilocarpine, physostigmine, neostigmine, chloroquine, amodiaquine, chloroguanide, primaquine, mefloquine, kinin, pridinol, prodipine, benztropine mesylate, trihexyphenidyl hydrochloride, ciprofloxacin, timolol, pindolol, quinacrine, benadryl, phatidyl hydrochloride, promethazine, dopamine, L-DOPA, serotonine, epinephrine, codeine, meperidine, methadone, morphine, atropin, decyclomine, methixene, propantheline, imipramine, amitriptyline, doxepin, desipramine, quinidine, proparanolol, lidocaine, chlorpromazine, promethazine, perphenazine, acridine orange, morphine and bupivacaine and combinations thereof.

[37] The method recited in any of [1] to [36], wherein the method is utilized for quality control of liposome preparations.

[38] The method recited in any of [1] to [36], wherein the method makes no use of a living body and/or living body component and is excellent in simplicity, correctness and reproducibility.

[39] The method recited in any of [1] to [36], wherein an in vivo pharmacokinetics is predictable in vitro.

[40] The method recited in any of [1] to [36], wherein an in vivo/in vitro correlation (IVIVC) is achieved.

### Advantageous Effect

According to the invention, the drug release characteristics of liposome preparations are directly measured simply without use of living bodies such as experiment animals and living body-derived substances such as blood serum and the like, thereby enabling accurate and highly reproducible test results to be obtained, along with IVIVC (in vivo/in vitro correlation) being achieved. Moreover, the method of the invention is able to confirm the releasability of produced liposome preparations on a lot-to-lot basis and can thus be favorably used as a quality control method of liposome preparations.

### BRIEF DESCRIPTION OF DRAWINGS

FIG. 1 is an image view of the release system of an entrapped drug using a pH gradient method as a remote loading method with ammonia as a shift reagent. The entrapped drug is released to an outer aqueous phase.

$$2 {NH}_{3} + \frac{2 ( Drug \text{-} {NH}_{3}^{+} ) - {SO}_{4}^{2}}{Entrapped drug} \to 2 {NH}_{4}^{+} + {SO}_{4}^{2 -} + \frac{2 Drug \text{-} {NH}_{2}}{Released drug} \qquad [ Chemical Formula 3 ]$$

FIG. 2 is a schematic view showing the detail of an entrapped drug release system in case where a pH gradient method is used as a remote loading method.

FIG. 3 is a flowchart showing a drug release testing method of the invention.

FIG. 4 is a graph showing a concentration of a shift reagent and a VCR release behavior.

FIG. 5 is a graph showing the relation between the concentration of a shift reagent and the VCR release rate constant.

FIG. 6 is a graph showing the relation between the pH of a shift reagent solution and the drug releasability.

FIG. 7 is a graph showing the influence of an osmotic pressure of a shift reagent solution on VCR release from liposomes.

FIG. 8 is a graph showing a VCR release behavior in a shift reagent solution containing an amino compound.

FIG. 9 is a graph showing a change in VCR release behavior depending on a phospholipid/cholesterol ratio of liposomes.

FIG. 10 is a graph showing drug release from liposomes made of phospholipids with different phase transition temperatures.

FIG. 11 is a graph showing releasability of DXR and VCR.

FIG. 12 is a graph showing a drug disposition of DXR and VCR in blood.

FIG. 13 is a graph showing releasability of VCR and CFX.

FIG. 14 is a graph showing releasability of DXR.

### MODE FOR CARRYING OUT THE INVENTION

Preferred embodiments of the invention are now described in detail.

### <Liposome-Membrane Structure>

In the invention, although a liposome means a closed vesicle formed of a phospholipid bilayer membrane, it may also mean, in some case, a liposome preparation that is a suspension containing such liposomes.

The liposome membrane structure of the invention is not critically limited, and may be any of unilamellar vesicles consisting of one membrane of the phospholipid bilayer membrane, multilammellar vesicles (MLV) or other structures. With the unilamellar vesicles, there may be any of SUV (small unilamellar vesicle), LUV (large unilamellar vesicles) or other structures.

### <Liposomes-Particle Size and EPR Effect>

The particle size of the liposomes of the invention is preferably set within a range where an EPR effect can be utilized. In more detail, the particle size of liposomes is preferably at 200 nm or below, more preferably at 50 to 200 nm. In this regard, where the use of the EPR effect is not required, no such limitation is placed on the size.

In the present specification, the term “EPR (Enhanced Permeability and Retention) effect” is used as having a meaning ordinarily employed in the art and means a phenomenon of enhancing vascular permeability in the vicinity of an inflamed area.

In general, it is known that where the EPR effect is permitted, particles having a size of about 200 nm or below can permeate a vascular wall (Int. J. Pharm. 1999, 190:49-56). Accordingly, when the particle size of liposomes is made at 200 nm or below, the transfer to a target cell can be achieved. Moreover, according to this EPR effect, liposomes can be continuously delivered to a target organ and a drug in the target organ arrives at a maximum blood concentration several hours delayed after administration and a delivered amount of the drug to the target organ is drastically increased (“Medical Application of Liposomes,” written by Lasic, D D and one other person). It will be noted that in cases where the liver is a target organ, no EPR effect is sought.

### <Liposome-Phospholipid Membrane>

With respect to phospholipids that are a main membrane material for the phospholipid membrane of liposomes of the invention, those phospholipids known to one in the art may be used singly or in plural combinations with their phase transition point being preferably higher than an internal body temperature (35 to 37° C.) and more preferably not lower than 40° C.

The phospholipids known to one in the art are amphipathic substances having a hydrophobic group made of a long-chain alkyl group and a hydrophilic group made of a phosphoric group in the molecule, and mention is made, for example, of: glycerophosphoric acids such as phosphatidylcholine (=lecithin), phosphatidylglycerol, phosphatidic acid, phosphatidylethanolamine, phosphatidylserine, phosphatidylinositol and the like; sphingophospholipids such as sphingomyelin (SM) and the like; natural or synthetic diphosphatidyl phospholipids and derivatives thereof such as cardiolipin and the like; and hydrogenated products thereof such as, for example, hydrogenated soybean phosphatidylcholine (HSPC) and the like.

Preferred phospholipids include hydrogenated phospholipids such as HSPC, etc., or SM etc., which have a phase transition temperature at which a drug entrapped in liposomes is not readily leaked out upon storage or in a living body such as blood.

<Liposomes-Membrane Components Other than Phospholipids>

The phospholipid membrane of the liposomes according to the invention may further include membrane components other than phospholipids in so far as liposomes can be stably formed.

The membrane components other than phospholipids include, for example, phosphoric acid-free lipids (other types of membrane lipids), membrane stabilizers, antioxidants and the like, if desired or if required.

Other types of lipids include, for example, fatty acids and the like.

The membrane stabilizers include, for example, sterols, such as cholesterol, glycerols and sugars such as sucrose, which are capable of lowering membrane fluidity.

The antioxidants include, for example, ascorbic acid, uric acid, tocopherol analogues, i.e. vitamin E and the like. Tocopherol includes four isomers of α, β, γ and δ forms, all of which are usable in the practice of the invention.

It will be noted that in the invention, the lipid of the liposome membrane component means all types of lipids other than drugs, such as a phospholipid of a main membrane substance, other type of membrane lipid, a lipid such as sterol or the like, which serves as the above-mentioned membrane stabilizer, and a lipid contained in a membrane modifier described hereinafter.

When the total amount of lipids of such membrane components is taken as 100 mol %, a phospholipid is contained preferably at 20 to 100 mol %, more preferably at 40 to 100 mol % and other types of lipids are contained preferably at 0 to 80 mol %, more preferably at 0 to 60 mol %.

In the practice of the invention, in so far as the membrane structure of liposomes are held, other membrane modifying components capable of being contained in liposome preparations can be contained within ranges not impeding the purpose of the invention.

### <Liposomes-Surface Modifying/Hydrophilic Macromolecules>

The liposomes in the invention may be modified on the surface of the phospholipid membrane thereof.

The membrane modification component includes hydrophilic macromolecules and other types of surface modifiers.

When the hydrophilic macromolecule is used as a lipid derivative thereof, a lipid moiety that is a hydrophobic moiety is held in the membrane, so that hydrophilic macromolecule chains can be stably distributed at an outer surface.

The hydrophilic macromolecules are not critical and include, for example, polyethylene glycol, polyglycerine, polypropylene glycol, ficoll, polyvinyl alcohol, a styrene-maleic anhydride alternate copolymer, a divinyl ether-maleic anhydride alternate copolymer, polyvinylpyrrolidone, polyvinyl methyl ether, polyvinyl methyloxazoline, polyethyloxazoline, polyhydroxypropyloxazoline, polyhydroxypropylmethacrylamide, polymethacrylamide, polydimethylacrylamide, polyhydroxypropyl methacrylate, polyhydroxyethyl acrylate, hydroxymethylcellulose, hydroxyethycellulose, polyaspartamide, synthetic polyamino acid and the like. Moreover, glucolipids are exemplified including water-soluble polysaccharides and derivatives thereof such as glucuronic acid, sialic acid, dextran, pullulan, amylose, amylopectin, chitosan, mannan, cyclodextrin, pectin, carrageenan and the like.

Preferred hydrophilic macromolecules include polyethylene glycol (PEG). This is because it has an effect of improving a blood retention although not limited to this reason.

The molecular weight of PEG is not critical and is preferably at 500 to 10,000 Da, more preferably at 1,000 to 7,000 Da and much more preferably at 2,000 to 5,000 Da.

As a lipid (hydrophobic moiety) of a hydrophilic macromolecule-lipid derivative, mention is made, for example, of a phospholipid, a long-chain fatty alcohol, a sterol, a polyoxypropylene alkyl, a glycerine fatty acid ester, or the like. In more detail, where PEG is used as a hydrophilic macromolecule, a phospholipid derivative or cholesterol derivative of PEG is mentioned. As the phospholipid, phosphatidylethanolamine is preferably mentioned, and as its acyl chain, mention is generally made of that of an about C₁₄ to C₂₀ saturated fatty acid, e.g. dipalmitoyl, distearoyl, palmitoylstearoyl or the like. For instance, a distearoylphosphatidylethanolamine derivative of PEG (PEG-DSPE) and the like are readily available general-purpose compounds.

In the liposome preparing step, although the timing of use of a membrane modification component is not critical, it is preferred that the membrane modification with a hydrophilic macromolecule is such that the hydrophilic macromolecule is selectively distributed at the outer surface of the liposome membrane, especially, at an external solution side from an outer membrane of the lipid bilayer membrane from the standpoint of a distribution efficiency and the unlikelihood of the hydrophilic macromolecule receiving an influence of a drug existing in an inner aqueous phase. Hence, according to the invention, it is preferred to add the component after formation of liposomes, especially, after a size-control step.

The liposome modification rate with a hydrophilic macromolecule, determined as a rate of an amount of a hydrophilic macromolecule relative to the membrane (total lipids), is preferably at 0.1 to 10 mol %, more preferably at 0.1 to 5 mol %.

### <Drug-Remote Loading Method>

The drug-entrapped liposomes of the invention are preferably prepared according to a remote loading method. Although the means of the remote loading method is not critical, there is exemplified a method making use of a citrate buffer solution or ammonium sulfate.

In the invention, the term “remote loading method” is used to indicate an ordinary meaning ordinarily known to one in the art, meaning a method to introduce a drug into liposomes by preparing empty liposomes wherein no drug is entrapped and adding a drug to an external solution of the liposomes.

In the remote loading method, the drug added to the external solution actively migrates to liposomes and entrapped in the liposomes. For this driving force, there is used a solubility gradient, an ion gradient, a pH gradient or the like. For instance, there is a method wherein an ion gradient established by separation with a liposome membrane is used to introduce a drug into liposomes. For instance, there is also a technique wherein a drug is added to the inside of liposomes preliminarily formed according to the remote loading method related to a Na⁺/K⁺ concentration gradient (Patent Document 3).

A proton concentration gradient is generally employed among ion gradients. For instance, there is mentioned such an embodiment to form the pH gradient using citric acid wherein the pH at the inside of liposomes (inner aqueous phase) is relatively lower than that at the outside of liposomes (outer aqueous phase). More particularly, the pH gradient can be formed such as by an ammonium ion gradient and/or a concentration gradient of an organic compound having an amino group capable of being protonated. In recent years, there is disclosed a method for carrying out a remote loading method by introducing an ionophore into a liposome membrane (U.S Published Application No. 2006/0193904)

### <Drug-Kind of Drug to be Entrapped>

The drug retained in drug-entrapped liposomes of the invention is not critically limited in so far as the drug can be entrapped into liposomes according to a remote loading method and it is retainable, and is preferably made of an amphipathic compound and more preferably made of an amphipathic, weakly basic compound.

The acid dissociation constant pKa of an amphipathic, weakly basic compound is preferably at 5 to 8.

Preferred examples of the amphipathic, weakly basic compound include epirubicin, daunorubicin, idarubicin, mitxanthrone, carcinomycin, N-acetyladriamycin, rubidazone, 5-imidodaunomycin, N-acetyldaunomycin, all anthracylin products, daunorylin, topotecan, 9-aminocamptotechin, 10,11-methylenedioxycamptotechin, 9-nitrocamptotechin, TAS103, 7-(4-methyl-piperadino-methylene)-10,11-ethylenedioxy-20(S)-camptotechin, 7-(2-isopropylamino)ethyl-20(S)-camptotechin, CKD-602, UCN-01, propranolol, pentamidine, dibucaine, bupivacaine, tetracaine, procaine, chlorpromazine, vinblastine, vincristine, vinorelbine, vindesine, mitomycin C, pilocarpine, physostigmine, neostigmine, chloroquine, amodiaquine, chloroguanide, primaquine, mefloquine, kinin, pridinol, prodipine, benztropine mesylate, trihexyphenidyl hydrochloride, ciprofloxacin, timolol, pindolol, quinacrine, benadryl, phatidyl hydrochloride, promethazine, dopamine, L-DOPA, serotonine, epinephrine, codeine, meperidine, methadone, morphine, atropin, decyclomine, methixene, propantheline, imipramine, amitriptyline, doxepin, desipramine, quinidine, proparanolol, lidocaine, chlorpromazine, promethazine, perphenazine, acridine orange, analgesic drugs such as morphine and bupivacaine, and the like.

Amphipathic acidic compounds are also preferred drugs, and preferred examples include steroid anti-inflammatory drugs such as prednisolone, methylprednisolone, dexamethasone and the like, non-steroid anti-inflammatory drugs (NSAIDs) such as aspirin, indomethacine, ibuprofen, felbinac, diclofenac, naproxen, mefenamic acid, phenylbutazone and the like, and angiotensin-converting enzyme (ACE) inhibitors such as captopril, benazepril, enalapril and the like.

### <Liposomes-Inner Aqueous Phase Solution>

With respect to the inner aqueous phase of liposomes used to entrap an amphipathic, weakly basic compound in the liposomes, selection of a counter ion is important.

In the invention, for instance, a counter ion to be entrapped in the liposomes along with an amphipathic, weakly basic drug can be selected from non-limitative examples including a hydroxide, a sulfate, a phosphate, a glucuronate, a citrate, a carbonate, a hydrogen carbonate, a nitrate, a cyanate, an acetate, a benzoate, a bromide, a chloride, other inorganic or organic anions, and anionic polymers, e.g. dextran sulfate, dextran phosphate, dextran borate, carboxymethyl dextran and the like.

The pH of the inner aqueous phase differs depending on the technique of the remote loading method. For instance, if citric acid is used, it is necessary to form a pH gradient between the inner aqueous phase and the outer aqueous phase beforehand. In that case, ΔpH is preferably 3 or over. With the other remote loading methods, no specific consideration is necessary since a pH gradient is formed by chemical equilibrium.

### <Liposomes-Outer Aqueous Phase Solution>

In the invention, the outer aqueous phase solution contains no shift reagent prior to mixing with a shift reagent. For example, there is contained no substance corresponding to a shift reagent exemplified as a deprotonating reagent and a conjugated acid thereof, and a protonating reagent and a conjugated base.

Where ammonia is used as a deprotonating reagent, the outer aqueous phase solution prior to mixing with a shift reagent preferably contains neither ammonia nor an ammonium ion.

In the outer aqueous phase solution, NaCl and/or sugars such as glucose or saccharose is preferably used as a solute.

The pH and osmotic pressure of the outer aqueous phase solution are preferably controlled by means of a buffer solution.

The pH is controlled preferably within a range of 5.5 to 7.5. This is because a pH difference at the time of decomposition of lipids and administration into a living body is taken into account, but not limited thereto. Nevertheless, in case where a pH gradient is established between the inside and outside of liposomes by use of citric acid, the pH is preferably in the vicinity of 7.0 as mentioned hereinbefore.

The osmotic pressure between the inner aqueous phase and outer aqueous phase of liposomes is not critical in so far as it is so controlled that the liposomes are not destroyed owing to the difference in osmotic pressure therebetween. When taking physical stability of liposomes into consideration, a smaller osmotic pressure difference between the inner aqueous phase and outer aqueous phase is preferred.

### <Shift Reagent>

The shift reagent used herein is a substance, which is considered to create an environment likely to release a drug from the inside of the liposomes by forming an ion gradient, opposite to an ion gradient formed upon entrapping of a drug, between the inner aqueous phase and the outer aqueous phase of the liposomes entrapping the drug therein, weakening an ion gradient formed to retain the drug, and causing a chemical equilibrium shift of the inner aqueous phase to weaken a retention of the drug retained as dissolved in the inner aqueous phase. For instance, deprotonating reagents and protonating reagents are exemplified.

In the invention, where a deprotonating reagent or protonating reagent is used as a shift reagent, it is considered that (1) the deprotonating reagent or protonating reagent permeates the phospholipids membrane of the liposomes in an non-ionized state and move from the outer aqueous phase to the inner aqueous phase; (2) in the inner aqueous phase of the liposomes, the deprotonating reagent serves as a Brønsted base and deprotonates the drug and the protonating reagent serves as a Brønsted acid and protonates the drug; and (3) consequently, the chemical equilibrium shift related to the drug in the inner aqueous phase of the liposomes is caused according to Le Chatelier's principle.

The deprotonating reagent is preferably made of ammonia or an amino compound, of which ammonia is more preferred.

The amino compound is preferably a low molecular weight amino compound having a molecular weight of not larger than 500. This is because lipid membrane permeability is taken into consideration, but not always limited thereto.

As to the amino group structure, mention is made of ammonia, a primary amine, a secondary amine and a tertiary amine.

As the deprotonating reagent, mention is made preferably of ammonia, methanolamine, ethanolamine, ethylenediamine, triethylamine and the like, of which ammonia is more preferred.

The shift reagent forming a deprotonating reagent in a buffer solution may be either a deprotonating reagent per se, or a salt made of a conjugated acid of the deprotonating reagent and an anion. Preferred examples of such anions include a hydroxide ion, a sulfate ion, a phosphate ion, a glucuronate ion, a citrate ion, a carbonate ion, a hydrogen carbonate ion, a nitrate ion, a cyanate ion, an acetate ion, a benzoate ion, a bromide ion, a chloride ion, and other inorganic or organic anions, and anions of anionic high molecular weight electrolytes such as dextran sulfate, dextran phosphate, dextran borate, carboxymethyl dextran and the like. Preferred examples of the salts of such conjugated acids and conjugated bases include a sulfate salt, a phosphate salt, a glucuronate salt, a citrate salt, a carbonate salt, a hydrogen carbonate salt, a nitrate salt, a cyanate salt, an acetate salt, a benzoate salt, a bromide salt, a chloride salt and other inorganic or organic salts, and salts of anionic high molecular weight electrolytes.

The shift reagent forming a protonating reagent in a buffer solution may be either a protonating reagent per se, or a salt of a conjugated base of a protonating reagent and a cation.

In a drug releasability test, there may be some cases where the concentration of a deprotonating reagent or protonating reagent influences the drug release rate. If the concentration is in excess, the release rate becomes too high and thus, a difficulty is involved in controlling the release rate, resulting in a difficulty in obtaining reproducible data. In contrast, if the concentration is too small, a satisfactory chemical equilibrium shift does not takes place, thus not arriving at possible measurement of a drug concentration. From this point of view, a final concentration of the shift reagent in a mixed system of a liposome preparation and a solution, to which the shift reagent has been added, is preferably at 0.1 to 150 mM.

In the drug releasability test, although the specific values of the pH and osmotic pressure of the mixed system of a liposome preparation and a solution, to which a shift reagent has been added, are not critical, simulation of living body environment conditions is advantageous in that it becomes possible to compare with releasability in a living body. From this viewpoint, it is preferred that the pH is in the range of 5.0 to 9.0 and the osmotic pressure is in the range of 20 to 400 mOsm, both in the mixed system. The pH and osmotic pressure are preferably controlled by means of a buffer solution.

### <Heating Temperature and Time of a Releasability Test>

In the drug release means and drug releasability evaluating method of the invention, the mixed system of a liposome preparation and a solution, to which a shift reagent has been added, can be heated at a give temperature for a given time thereby promoting the release.

This temperature should be determined while taking a phase transition temperature in the lipid of liposomes into consideration and can be changed depending on the lipid composition of the liposomes. In addition, in view of the advantage in that simulation of body environment conditions makes it possible to compare with the releasability in a living body for the reason set out above, it is preferred that the temperature is chosen from temperatures in the vicinity of a body temperature, e.g. within a range, for example, of 30 to 40° C. Nevertheless, because the temperature should be set depending on the types of drug and shift reagent and the lipid composition, the temperature is not specifically critically.

The given time is preferably set at a time after transition of from an initial release rate to a stable release rate and is also set preferably at a time that involves the smallest error in respect of a testing method.

When taking a quality control method in the course of manufacture into consideration, too long a time is not favorable and the time is preferably set within a range of 1 to 180 minutes although not limited to this range because of the necessity of the time setting depending on the types of drug and shift reagent and the lipid composition.

### <Stop Solution>

In the drug release means and drug releasability evaluating method of the invention, the mixed system of a liposome preparation and a solution, to which a shift reagent has been added, is heated at a given temperature for a given time thereby enabling the release to be promoted.

The release can be stopped by adding a coolant or stop solution.

The stop solution means a solution capable of stopping the release from liposomes and is characterized in that no shift reagent is added.

The addition of a stop solution means to dilute a concentration of the added shift reagent and thus, a once released drug may be again entrapped in the liposomes. The role of the stop solution is to inhibit the release of the drug brought about by means of the added shift reagent. In view of such concern, it is preferred that the pH of the stop solution is so set that in order to reduce a pH gradient between the inner and outer aqueous phases of the liposomes and take the pKa of the added shift reagent into consideration, the permeability of the added shift reagent through the liposome membrane can be remarkably lowered. More particularly, the pH is preferably within a range of 1.0 to 5.0. The pH is preferably controlled by use of a buffering reagent. The osmotic pressure of the stop solution should preferably be as close as to an osmotic pressure of the mixed system of a liposome preparation and a solution, to which a shift reagent has been added, because an influence of the difference in osmotic pressure can be eliminated.

It is known that liposomes so behave at a phase transition temperature or over of the lipid that diffusion of a drug toward outside of the liposomes is accelerated. The purpose of the stop solution is to stop the diffusion of the drug, so that the temperature of the stop solution is preferably as low as possible, and a specific example that is feasible is ice cooling.

### <Quantitative Determination of a Released Drug>

In the drug releasability evaluating method of the invention, it is preferred to separate a released drug from liposomes.

For a method of separating the drug, there may be used a variety of methods known to one in the art without limitation. Examples of the method of separating a drug preferably include a dialysis membrane, gel permeation chromatography, a filtration method using a filter, a method of a flow-through cell that is one of ultracentrifuges and elution testing devices, and the like.

In the drug releasability evaluating method of the invention, after separation of a released drug, the quantitative determination of the drug enables a release amount and release rate to be calculated. As to the quantitative analysis of a drug, testing methods known to one in the art may be used and preferred examples include a quantitative determination method using a UV spectrophotometer and a fluorospectrophotometer, a high-performance liquid chromatograph HPLC), and the like.

### <Container or Device for Carrying Out a Release Test>

The container or device used in the drug release test of the invention is not critical and the use of from microtubes to existing elution testers is possible.

It will be noted that where there is used a container or device preliminarily equipped with the function to separate the released drug and the drug entrapped into liposomes from each other, e.g. a dialysis membrane, a flow-through cell or the like, no stop solution above-mentioned may be added.

### EXAMPLES

Examples are described below to more particularly illustrate the invention. It is as a matter of course that the invention should not be construed as limited to these examples.

The stop solution preparation method, lipid concentration measurement method, particle size measurement method, drug determination method and release rate calculation method in these supplemental examples are set forth below.

### <Stop Solution Preparation Method>

5.84 g of sodium chloride and 15.60 of dihydrogen sodium phosphate were dissolved in 900 ml of water. Next, a phosphate buffer solution was added to the solution to adjust the pH to 3.0, followed by further addition of water to make 1000 ml in total volume.

This was provided as a stop solution (which may be referred to herein as “release stop solution”).

It will be noted that the osmotic pressure of the stop solution prepared according to the above preparation procedure was at 300 mOsm.

### <Lipid Concentration Measurement Method>

Using a phospholipid quantitative determination kit (Phospholipid C Test Wako, made by Wako Pure Chemical Industries, Ltd.), a concentration (unit: mg/ml) of a phospholipid (HSPC, DPPC, DMPC or DSPC) in a liposome dispersion was measured.

### <Particle Size Measurement Method>

20 μl of a liposome dispersion was diluted with 3 ml of a physiological saline solution. Using Zetasizer 3000 HS (made by Malvern Instruments Ltd.), an average particle size (unit: nm) was measured according to photon correlation spectroscopy.

### <Quantitative Determination Method of Drug>

### (1) Quantitative Determination Method of Vincristine (VCR)

100 μl of VCR-containing liposomes prepared in a manner as described in Preparatory Example described hereinafter was dispersed in 2 ml of methanol to provide a sample solution.

Separately, 100 μl of a VCR aqueous solution having a different concentration was taken out and dispersed in 2 ml of methanol to prepare a reference solution for calibration curve.

These solutions were subjected to measurement by a HPLC method under measuring conditions indicated below.

The VCR concentration was calculated according to a calibration curve equation.

### Measuring Conditions

Column: C8 column (250×4.6 mm, 5 μm)

Measuring wavelength: 298 nm

Mobile phase: a solution prepared by adding phosphoric acid to 700 ml of water/ethylenediamine (59/1) so as to adjust the pH to 7.5): methanol=700:300

Flow rate: 1 ml/minute

Injected amount: 10 μl

Column temperature: 40° C.

### (1.1) Quantitative Determination Method of VCR in an External Solution

VCR-containing liposomes prepared in a manner as described in Preparatory Example described hereinafter was subjected to 1:10 dilution with a physiological saline solution, followed by separating the drug, not contained in the liposomes, by ultracentrifugation to provide a sample solution. Separately, 100 μl of a VCR aqueous solution having a different concentration was taken out, to which 900 μl of water was added, thereby preparing a reference solution for calibration curve. The sample solution and reference solution were subjected to quantitative determination under the HPLC conditions indicated with respect to the VCR determination.

### (1.2) Quantitative Determination Method of Released VCR

After 1:10 dilution of the sample with a stop solution, the drug, not contained in the liposomes, was separated by ultracentrifugation to provide a sample solution. Separately, 100 μl of a VCR aqueous solution having a different concentration was taken out, to which 900 μl of water was added. Moreover, this solution was diluted with water at 1:10 to provide a reference solution for calibration curve. The sample solution and reference solution were subjected to quantitative determination under the HPLC conditions indicated before with respect to the VCR determination except that an injected amount was at 50 μl only in this determination method.

### (2) Quantitative Determination Method of Ciprofloxacin (CFX)

100 μl of CFX-containing liposomes prepared in a manner as described in Preparatory Example described hereinafter was dispersed in 2 ml of methanol to provide a sample solution. Separately, 100 μl of a CFX aqueous solution having a different concentration was taken and dispersed in 2 ml of methanol to prepare a reference solution for calibration curve. These solutions were subjected to measurement by the HLPC method under measuring conditions indicated below. The CFX concentration was calculated according to a calibration curve equation.

### Measuring Conditions

Column: C8 column (250×4.0 mm, 5 μm)

Measuring wavelength: 278 nm

Mobile phase: 130 ml of acetonitrile was added to 870 ml of a 0.02 mol/l phosphate buffer solution having a pH of 3.0.

Flow rate: 1 ml/minute

Injected amount: 10 μl

Column temperature: 40° C.

### (2.1) Quantitative Determination Method of CFX in an External Solution

CFX-containing liposomes prepared in a manner as described in Preparatory Example described hereinafter was diluted with a physiological saline solution at 1:10, followed by separating a drug, not contained in the liposomes, by ultracentrifugation to provide a sample solution. Separately, 100 μl of a CFX aqueous solution having a different concentration was taken out, to which 900 μl of water was added thereby preparing a reference solution for calibration curve. The sample solution and reference solution were quantitatively determined under the HPLC conditions indicated with respect to the CFX determination.

### (2.2) Quantitative Determination Method of Released CFX

After dilution of the sample with a stop solution at 1:10, the drug, not contained in the liposomes, was separated by ultracentrifugation to provide a sample solution. Separately, 100 μl of a VCR solution having a different concentration was taken out, to which 900 μl of water was added. Moreover, this solution was diluted with water at 1:10 to provide a reference solution for calibration curve. The sample solution and reference solution were subjected to quantitative determination under the HPLC conditions indicated with respect to the CFX determination.

### (3) Quantitative Determination Method of Doxorubicin (DXR)

0.5 ml of DXR-containing liposomes prepared in a manner as described in Preparatory Example described hereinafter was dispersed in 20 ml of methanol. 1 ml of a 0.1 mol/l dihydrogen sodium phosphate solution having a pH of 3.0 was added to and mixed with 1 ml of the solution to provide a sample solution. Separately, 1 ml of a 0.1 mol/l dihydrogen sodium phosphate solution having a pH of 3.0 was added to and mixed with 1 ml of a DXR solution having a different concentration to prepare a reference solution for calibration curve. The sample solution and reference solution were subjected to measurement according to the HPLC method under measuring conditions indicated below. The DXR concentration was calculated according to a calibration curve equation.

### Measuring Conditions

Guard column: GL cart Inertsil ODS-2 (made by GL Science Co., Ltd.)

Column (Inertsil ODS-2 (250×4.6 mm, 5 μm) (made by GL Science Co., Ltd.)

Measuring wavelength: 254 nm

Mobile phase: a solution obtained by adding 900 ml of RO water to 5 ml of formic acid and adjusted in pH to 4.0 by means of ammonia water:acetonitrile=700:300.

Flow rate: 1 ml/minute

Injected amount: 50 μl

Column temperature: 40° C.

### (3.1) Quantitative Determination Method of DXR in an External Solution

After dilution of DXR-containing liposomes, prepared in a manner as described in Preparatory Example described hereinafter, with a physiological saline solution at 1:10, the drug, not contained in the liposomes, was separated by ultracentrifugation. After the ultracentrifugation, 50 μl of a supernatant of the sample was dispersed in 2 ml of methanol for fluorescent analysis to provide a sample solution. Separately, 50 μl of a DXR aqueous solution having a different concentration was dispersed in 2 ml of methanol for fluorescent analysis to prepare a reference solution for calibration curve. The sample solution and reference solution were subjected to quantitative determination of fluorescent intensities at excitation wavelengths of 480 nm and a fluorescence wavelength of 580 nm by use of a fluorospectrophotometer.

### (3.2) Quantitative Determination Method of Released DXR

After dilution of the sample with a stop solution at 1:10, the drug, not contained in the liposomes, was separated by ultracentrifugation. 1 ml of a 0.1 mol/l dihydrogen sodium phosphate solution having a pH of 3.0 was added to and mixed with 1 ml of a supernatant of the sample obtained after the ultracentrifugation to provide a sample solution. Separately, 1 ml of a 0.1 mol/l dihydrogen sodium phosphate solution having a pH of 3.0 was added to and mixed with 1 ml of/a DXR solution having a different concentration to prepare a reference solution for calibration curve. The sample solution and reference solution were subjected to measurement under HPLC conditions indicated with respect to the DXR determination.

### <Calculation Method of a Release Rate>

The drug release rate (unit: %) was calculated according to the following equation.

Release rate (%)=amount of released drug/total drug amount×100

Abbreviations and molecular weights of drugs, etc., used in examples are indicated below.

HSPC: Hydrogenated soybean phosphatidyl choline (molecular weight: 790, made by Lipoid GmbH)

DPPC: Dipalmitoylphosphatidyl choline (molecular weight: 734.15, made by NOF Corporation)

DSPC: Distearoylphosphatidyl choline (molecular weight: 790.15, made by NOF Corporation)

DMPC: Dimyristoylphosphatidyl choline (molecular weight: 677.94, made by NOF Corporation)

PEG5000-DSPE: Polyethylene glycol (molecular weight 5000)-phosphatidyl ethanolamine (molecular weight 6081, made by NOF Corporation)

Chol.: Cholesterol (molecular weight: 386.86, made by Solvay Co., Ltd.)

VCR: Vincristine sulfate (molecular weight: 923,04, made by Changzhou LEO Chemical Co., Ltd.)

CFX: Ciprofloxacin (molecular weight: 331.34, made by Zhejiang Jiashan Chengda Pharm. & Chem. Co., Ltd.)

DXR: Doxorubicin hydrochloride (molecular weight: 579.98, made by RPG Life Sciences Ltd.)

2-aminoethanol (molecular weight: 61.08, made by Kanto Chemical Co., Inc.)

Diethylamine (molecular weight: 73.14, made by Kanto Chemical Co., Inc.)

Ethylenediamine (molecular weight: 60.10, made by Kanto Chemical Co., Inc.)

### Preparatory Example 1

### Preparation of VCR-Containing Liposomes

### 1. Preparation of VCR-Containing Liposomes

VCR-containing liposomes were prepared according to the following steps.

### (1) Preparation of a Lipid Dispersion

0.71 g of HSPC and 0.29 g of Chol. were, respectively, weighed. These were mixed with 1 ml of absolute ethanol, followed by heating and dissolution in a thermostatic oven at 70° C. to obtain an ethanol solution of the lipids. A 250 mM citric acid aqueous solution and sucrose were added to the ethanol solution to make the osmotic pressure of the ethanol solution at 500 mOsm. Next, 9 ml of a liquid whose pH was adjusted to 2 was added to the ethanol solution, followed by further heating to obtain a lipid dispersion.

### (2) Size-Control of Liposomes

The thus obtained lipid dispersion was subjected to size-control by passing through a two-stacked filter (0.1 μm, polycarbonate membrane, made by Whatman Co., Ltd.) attached to an Extruder T.10 (Lipex Biomembranes, Ltd.) heated to about 70° C., thereby obtaining a suspension of the liposomes after the size-control.

### (3) Surface Modification of the Liposomes

A PEG5000-DSPE aqueous solution (concentration: about 0.04 g/ml) was provided in such an amount that a content of the PEG5000-DSPE corresponded to 0.75 mol % of the afore-weighed total lipid amount (a sum of HSPC and Chol.). This PEG5000-DSPE aqueous solution was heated in a thermostatic oven preliminarily set at 65° C. This PEG5000-DSPE aqueous solution and the suspension of the liposomes after the size-control were mixed together. After the mixing, the mixture was heated in the thermostatic oven set at 65° C. for further 30 minutes to obtain a PEG-modified liposome suspension.

### (4) Replacement of an External Solution

A gel column (Sephalose 4 Fast Flow, made by Amersham Biosciences Co.) wherein the mobile phase was replaced by a 10% sucrose/10 mM histidine solution (pH: 6.5) was provided. Using this gel column, an external solution of the suspension of the PEG-modified liposomes was substituted with the 10% sucrose/10 mM histidine solution (pH: 6.5) to obtain a suspension of the liposomes after the replacement of the external solution. By the replacement of the external solution, a pH gradient was established between the inside and outside of the liposome membrane.

### (5) Entrapping of a Drug

A VCR aqueous solution was added to the suspension of the liposomes after the replacement of the external solution in such a way that a ratio by weight of VCR and HSPC (VCR/HSPC) was given 0.14 (w/w). This was heated in a thermostatic oven at 60° C. for 30 minutes to permit the drug to be entrapped, thereby obtaining a suspension of the drug-entrapped liposomes.

### (6) Elimination of a Non-Entrapped Drug

A gel column (Sephalose 4 Fast Flow) wherein the mobile phase was replaced by a 10% sucrose/10 mM histidine solution (pH: 6.5) was provided. Using this gel column, the 10% sucrose/10 mM histidine solution (pH: 6.5) was employed as a mobile phase to eliminate the drug left in the external solution of the suspension of the drug-entrapped liposomes to obtain a suspension of liposomes after elimination of the non-entrapped drug.

Finally, the suspension was filtrated with a filter (0.2 μm) to obtain liposomes after the filtration with the filter.

### 2. Liposome Characteristics of VCR-Containing Liposomes

In Table 1, there are shown the VCR concentration and lipid concentration of the liposomes (hereinafter abbreviated as “LIP1”) after the elimination of the non-entrapped drug, and the VCR concentration and particle size of the liposomes after the filtration with the filter, each prepared according to the Preparatory Example 1. It will be noted that the measurement of the VCR concentration (quantitative determination of VCR), and the measurements of the lipid concentration and particle size were, respectively, made according to the methods set out hereinbefore.

TABLE 1
Liposome characteristics

| | | Liposomes | | Liposomes | |
|---|---|---|---|---|---|
| | | after elimination of | | after filtration | |
| | | non-entrapped drug | | with filter | |
| | | VCR | | VCR | |
| | | concen- | Lipid | concen- | |
| | HSPC/Chol. | tration | concentration | tration | Particle |
| | (ratio by weight) | (mg/ml) | (mg/ml) | (mg/ml) | size (nm) |
| LIP1 | 54/46 | 1.49 | 1.37 | 1.37 | 113.9 |

### Example 1

### Influence of a Shift Reagent Concentration on Drug Releasability

In this example, the influence of the concentration of a shift reagent on the release of from drug-entrapped liposomes to an external solution of liposomes was examined. It will be noted that in this example, ammonium acetate was used as a shift reagent.

### 1. Preparation of a Shift Reagent Solution

Ammonium acetate was dissolved so as to make ammonium ion concentrations of 5, 20, 25, 50 and 100 mM and the pH of the solutions was adjusted to 7.0 by use of a 0.1 M sodium hydroxide test solution. Moreover, sodium chloride was added to the solutions so that an osmotic pressure thereof was made at 300 mOsm, thereby providing shift reagent solutions.

### 2. Quantitative Determination of Released VCR

The liposomes shown in Preparatory Example 1 were diluted with the shift reagent solution at 1:10 and heated to 37° C. 2.5, 5, 10, 15 and 30 minutes after commencement of the heating, samples were taken out. It is to be noted that the samples were stored under ice cooling before use. The quantitative determination of released VCR was made according to the method described hereinbefore.

### 3. Results

In FIG. 4, there are shown the results of the examination of the influence of the shift reagent concentration in the shift reagent solution related to the VCR release from the VCR-entrapped liposomes. As a consequence, it has been revealed that the release of VCR increases with time for all the shift reagent solutions having different shift reagent concentrations and that the release amount of VCR increases dependently on the shift reagent concentration.

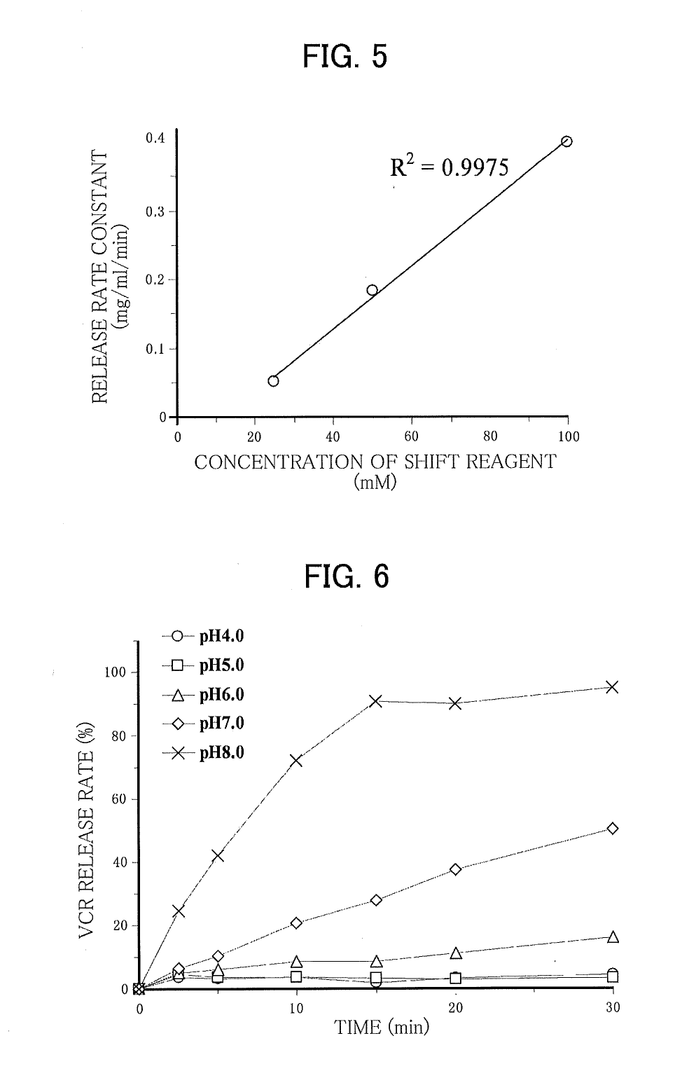

FIG. 5 shows the relationship between the release rate constant and the shift reagent concentration.

It has been demonstrated that since the shift reagent concentration and the release rate constant have high correlation (r²=0.9975) with each other, the drug release from the liposomes depends on the shift reagent concentration and can be readily controlled by controlling the shift reagent concentration.

### Example 2

### Relation Between the pH of a Shift Reagent Solution and Drug Releasability

In this example, there was examined the influence of the pH of a shift reagent solution on the release of from drug-bearing liposomes to an external solution of the liposomes. It will be noted that in this example, ammonium acetate was used as a shift reagent.

### 1. Preparation of a Shift Reagent Solution

Ammonium acetate was dissolved so as to make an ammonium ion concentration of 50 mM, followed by controlling the pH of the solution at 4.0, 5.0, 6.0, 7.0 and 8.0 by use of a 1 M sodium hydroxide test solution. Moreover, sodium chloride was added to the solution to make an osmotic pressure of 300 mOsm thereby providing a shift reagent solution.

### 2. Quantitative Determination of Released VCR

The liposomes shown in Preparatory Example 1 were diluted with the shift reagent solution at 1:10 and heated at 37° C. Sampling was carried out 2.5, 5, 10, 15 and 30 minutes after commencement of the heating. It will be noted that the samples were stored under ice cooling before use. The released VCR was quantitatively determined by the method set out hereinbefore.

### 3. Results

FIG. 6 shows a change in release behavior of VCR when the pH of the shift reagent solution is set at 4.0, 5.0, 6.0, 7.0 or 8.0.

It has been made clear that the release behavior of VCR depends on the pH of the shift reagent solution and that in this example, the releasability of VCR is promoted with increasing pH.

### Example 3

### Relation Between the Osmotic Pressure of Shift Reagent Solution and Drug Releasability

In this example, there was examined the influence of the osmotic pressure of a shift reagent solution on the release of from drug-entrapped liposomes to an external solution of the liposomes. It will be noted that in this example, ammonium acetate was used as a shift reagent.

### 1. Preparation of a Shift Reagent

Ammonium acetate was so dissolved as to make an ammonium ion concentration of 50 mM with its pH being adjusted to 7.0. Sodium chloride was added to the solution so that an osmotic pressure thereof was controlled at from 100 mOsm to 750 mOsm, thereby proving a shift reagent solution.

### 2. Quantitative Determination of Released VCR

The liposomes shown in Preparatory Example 1 were diluted with the shift reagent solution at 1:10 and heated at 37° C. Samples obtained by sampling 2.5, 5, 10, 15 and 30 minutes after commencement of the heating were taken out. It will be noted that the samples were kept under ice cooling before use. The quantitative determination of the released VCR was made according to the method set out hereinbefore.

### 3. Results

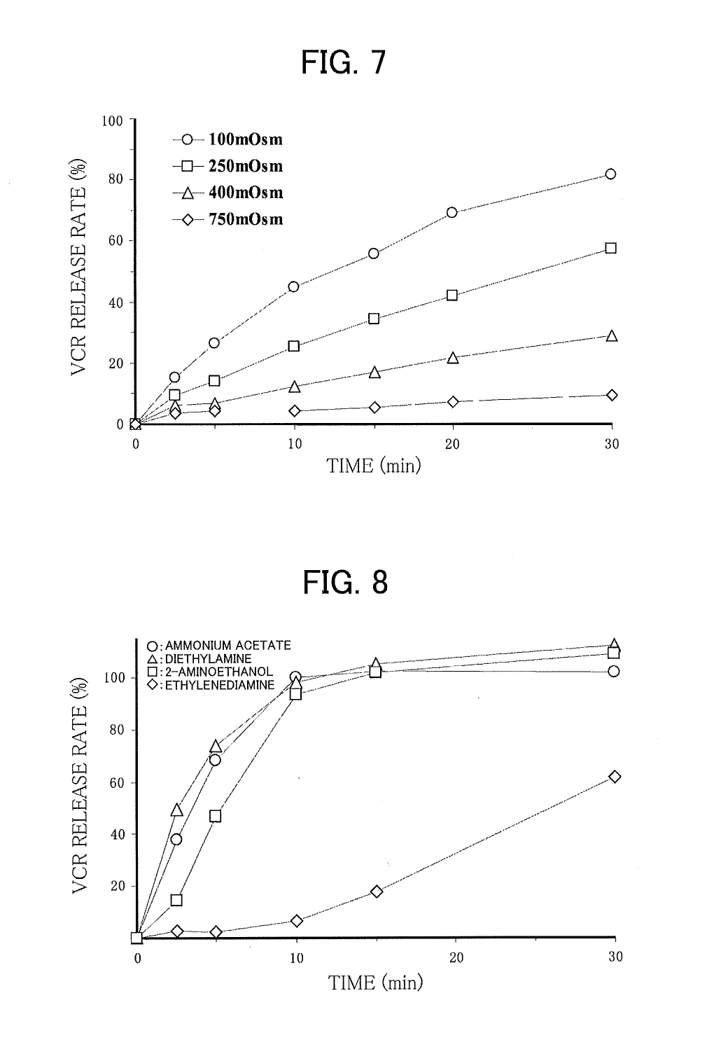

FIG. 7 is a view showing the influence of the osmotic pressure of the shift reagent solution on the VCR release from the liposomes.

The release rate of VCR is greatly influenced by the osmotic pressure and it has been revealed that a smaller osmotic pressure of the shift reagent solution leads to more promoted release of VCR.

### Preparatory Example 2

### Preparation of VCR-Containing Liposomes

### 1. Preparation of VCR-Containing Liposomes

VCR-containing liposomes were prepared according to the following steps.

### (1) Preparation of a Lipid Dispersion

0.71 g of HSPC and 0.29 g of Chol. were, respectively, weighed. These were mixed with 1 ml of absolute ethanol, followed by heating and dissolution in a thermostatic oven at 70° C. to obtain an ethanol solution of the lipids. A 250 mM citric acid aqueous solution and sucrose were added to the ethanol solution to control the osmotic pressure of the ethanol solution at 500 mOsm. Next, 9 ml of a liquid whose pH was adjusted to 2.5 was added to the ethanol solution, followed by further heating to obtain a lipid dispersion.

### (2) Size-Control of Liposomes

The thus obtained lipid dispersion was subjected to size-control by passing through a two-stacked filter (0.1 μm, polycarbonate membrane, made by Whatman Co., Ltd.) attached to an Extruder T.10 (Lipex Biomembranes, Ltd.) heated to about 70° C. and thus, a suspension of liposomes after the size-control was obtained.

### (3) Surface Modification of the Liposomes

A PEG5000-DSPE aqueous solution (concentration: about 0.04 g/ml) was provided in such an amount that a content of the PEG5000-DSPE corresponded to 0.75 mol % of the afore-weighed total lipid amount (a sum of HSPC and Chol.). This PEG5000-DSPE aqueous solution was heated in a thermostatic oven preliminarily set at 65° C. The PEG5000-DSPE aqueous solution and the suspension of the liposomes after the size-control were mixed together. After the mixing, the mixture was heated in the thermostatic oven set at 65° C. for further 30 minutes to obtain a PEG-modified liposome suspension.

### (4) Replacement of an External Solution

A gel column (Sephalose 4 Fast Flow, made by Amersham Biosciences Co.) wherein the mobile phase was replaced by a 10% sucrose/10 mM histidine solution (pH: 6.5) was provided. Using this gel column, an external solution of the suspension of the PEG-modified liposomes was substituted with the 10% sucrose/10 mM histidine solution (pH: 6.5) to obtain a suspension of the liposomes after the replacement of the external solution. By the replacement of the external solution, a pH gradient was established between the inside and outside of the liposome membrane.

### (5) Entrapping of a Drug

A VCR aqueous solution was added to the suspension of the liposomes after the replacement of the external solution in such a way that a ratio by weight of VCR and HSPC (VCR/HSPC) was given 0.22 (w/w). This was heated in a thermostatic oven at 60° C. for 30 minutes to permit the drug to be entrapped, thereby obtaining a suspension of the drug-entrapped liposomes.

### (6) Elimination of a Non-Entrapped Drug

A gel column (Sephalose 4 Fast Flow) wherein the mobile phase was replaced by a 10% sucrose/10 mM histidine solution (pH: 6.5) was provided. Using this gel column, the 10% sucrose/10 mM histidine solution (pH: 6.5) was employed as a mobile phase to eliminate the drug left in the external solution of the suspension of the drug-entrapped liposomes to obtain a suspension of the liposomes after the elimination of the non-entrapped drug.

Finally, the suspension was filtrated with a filter (0.2 μm) to obtain liposomes after the filtration with the filter.

### 2. Liposome Characteristics of VCR-Containing Liposomes

In Table 2, there are shown liposome characteristics of the VCR-containing liposomes (hereinafter abbreviated as “LIP2”) prepared according the Preparatory Example 2. It will be noted that the measurement of a drug concentration in the liposomes (quantitative determination of VCR) and the measurement of a particle size were carried out according to the methods set out hereinbefore, respectively.

TABLE 2
Liposome characteristics

| | | Drug | |
|---|---|---|---|
| | | concentration | Particle size |
| | Drug | (mg/ml) | (nm) |
| LIP2 | VCR | 1.29 | 114.6 |

### Example 4

### VCR Releasability in an Amino Compound-Containing Shift Reagent Solution

In this example, a VCR release behavior is shown when using, as a shift reagent, primary amines (2-aminoethanol, ethylenediamine) and a secondary amine (diethylamine). It will be noted that ammonium acetate that is an ammonium salt was used as a reference shift reagent and LIP2 was used as liposomes.

### 1. Preparation of a Shift Reagent Solution

A shift reagent was weighed, to which a phosphate buffer solution was added thereby obtaining a 50 mM shift reagent solution having a pH of 7.4.

### 2. Quantitative Determination of Released VCR

The liposomes were diluted at 1:10 by addition of the shift reagent solution and heated at 37° C. Sampling was carried out 2.5, 5, 10, 15 and 30 minutes after commencement of the heating. It will be noted that the samples were stored under ice cooling before use. The released VCR was subjected to quantitative determination according to the method set out hereinbefore.

### 3. Results

FIG. 8 is a view showing an influence of the shift reagents in the VCR release from the liposomes.

Although the release behavior differs by use of the amino compounds, it has been made clear that VCR can be released, revealing that aside from ammonia, amino compounds can be chosen as a shift reagent in this release test.

### Conclusion of Examples 1 to 4

As shown in Examples 1 to 4, it has become evident that the drug release from liposomes is influenced by the solution characteristics of the shift reagent solution. More particularly, it has been suggested that the drug release from liposomes greatly differs depending on the external environment. From this point of view, it has been suggested that in case where drug releasability is assessed with an in vivo basis in mind, solution characteristics should be preferably so controlled as to come close to a living body.

### Preparatory Example 3

### Preparation of VCR-Liposomes Having Different Membrane Physical Properties

### 1. Preparation of VCR-Containing Liposomes

The VCR-containing liposomes having different membrane physical properties were prepared according to the following steps.

### (1) Preparation of a Lipid Dispersion

PC¹ (HSPC, DSPC, DPPC or DMPC) and Chol. were, respectively, weighed in amounts (unit: g) indicated in Table 3. These were mixed with 1 ml of absolute ethanol and heated for dissolution in a thermostatic oven at 70° C. to obtain an ethanol solution of the lipids. A citric acid aqueous solution having a concentration of 250 mM was added to this ethanol solution thereby adjusting the pH of the ethanol to 3.0. Sodium chloride was added to the solution to adjust the osmotic pressure of the ethanol solution at 500 mOsm. Moreover, this ethanol solution was heated in the thermostatic oven set at 70° C. to obtain a lipid dispersion.

TABLE 3
Formulations of lipid membranes and weighed
amounts

| | | | Weighed value |
|---|---|---|---|
| | | | [g] |
| | | | Top: PC¹ |
| | PC¹ | PC¹/Chol. | Bottom: Chol. |
| LIP3 | HSPC | 54/46 | 0.71 |
| | | | 0.29 |
| LIP4 | HSPC | 65/35 | 0.85 |
| | | | 0.22 |
| LIP5 | DSPC | 54/46 | 0.71 |
| | | | 0.29 |
| LIP6 | DPPC | 54/46 | 0.69 |
| | | | 0.31 |
| LIP7 | DMPC | 54/46 | 0.67 |
| | | | 0.23 |

PC¹: Phospholipid (HSPC, DSPC, DPPC or DMPC)

### (2) Size-Control of Liposomes

The thus obtained lipid dispersion was subjected to size-control by passing through a two-stacked filter (0.1 μm, polycarbonate membrane, made by Whatman Co., Ltd.) attached to an Extruder T.10 (Lipex Biomembranes, Ltd.) heated to about 70° C. and thus, a suspension of the liposomes after the size-control was obtained.

### (3) Surface Modification of the Liposomes

A PEG5000-DSPE aqueous solution (concentration: about 0.04 g/ml) was provided in such an amount that a content of the PEG5000-DSPE corresponded to 0.75 mol % of the afore-weighed total lipid amount (a sum of PC¹ and Chol.). This PEG5000-DSPE aqueous solution was heated in a thermostatic oven preliminarily set at 65° C. The PEG5000-DSPE aqueous solution and the suspension of the liposomes after the size-control were mixed together. After the mixing, the mixture was heated in the thermostatic oven set at 65° C. for further 30 minutes to obtain a PEG-modified liposome suspension.

### (4) Replacement of an External Solution

A gel column (Sephalose 4 Fast Flow, made by Amersham Biosciences Co.) wherein the mobile phase was replaced by a 10% sucrose/10 mM histidine solution (pH: 6.5) was provided. Using this gel column, an external solution of the suspension of the PEG-modified liposomes was substituted with the 10% sucrose/10 mM histidine solution (pH: 6.5) to obtain a suspension of the liposomes after the replacement of the external solution. By the replacement of the external solution, a pH gradient was established between the inside and outside of the liposome membrane.

### (5) Entrapping of a Drug

The phospholipid contained in the liposomes after the replacement of the external solution was quantitatively determined by use of a phospholipid measuring kit. A VCR aqueous solution was added to the suspension of the liposomes after the replacement of the external solution in such a way that a value of VCR/PC¹ relative to the total lipid concentration calculated from the results of the phospholipid quntitative deteremination was made at 0.10 mol/mol. This was heated in a thermostatic oven at 60° C. for 30 minutes to permit the drug to be entrapped, thereby obtaining a suspension of the drug-entrapped liposomes.

### (6) Elimination of a Non-Entrapped Drug

A gel column (Sephalose 4 Fast Flow) wherein the mobile phase was replaced by a 10% sucrose/10 mM histidine solution (pH: 6.5) was provided. Using this gel column, the 10% sucrose/10 mM histidine solution (pH: 6.5) was employed as a mobile phase to eliminate the drug left in the external solution of the suspension of the drug-entrapped liposomes to obtain a suspension of liposomes after the elimination of the non-entrapped drug.

Finally, the suspension was filtrated with a filter (0.2 μm) to obtain liposomes after the filtration with the filter.

### 2. Liposome Characteristics of the VCR-Containing Liposomes

In Table 4, there are shown the liposome characteristics of the VCR-containing liposomes (hereinafter abbreviated as “LIP3,” “LIP4,” “LIP5,” “LIP6” or “LIP7”) having different membrane physical properties and prepared according to the Preparatory Example 3.

TABLE 4
Liposome characteristics

| | | | VCR | |
|---|---|---|---|---|
| | | | concentration | PC¹ concentration |
| | PC¹ | PC¹/Chol. | (mg/ml) | (mg/ml) |
| LIP3 | HSPC | 54/46 | 0.96 | 7.9 |
| LIP4 | HSPC | 65/35 | 0.94 | 9.8 |
| LIP5 | DSPC | 54/46 | 1.1 | 8.3 |
| LIP6 | DPPC | 54/46 | 0.93 | 7.1 |
| LIP7 | DMPC | 54/46 | 0.79 | 6.8 |

PC¹: Phospholipid (HSPC, DSPC, DPPC or DMPC)

### Example 5

### Relation Between Liposome Membrane Composition and Drug Releasability

In this example, an investigation was made using LIP3 to 7 liposomes.

### 1. Preparation of a Shift Reagent Solution

Ammonium acetate was dissolved in a phosphate buffer solution having a pH of 7.4 in such a way that an ammonium ion concentration was made at 50 mM. Moreover, sodium chloride was added to the solution to adjust the osmotic pressure at 300 mOsm. The resulting solution was used as a shift reagent solution.

### 2. Quantitative Determination of Released VCR

Liposomes were diluted with the shift reagent solution at 1:10 and heated at 37° C. Sampling was carried out 2.5, 5, 10, 15 and 30 minutes after commencement of the heating. It will be noted that the samples were stored under ice cooling before use. The released VCR was quantitatively determined by the method set out hereinbefore.

### 3. Results

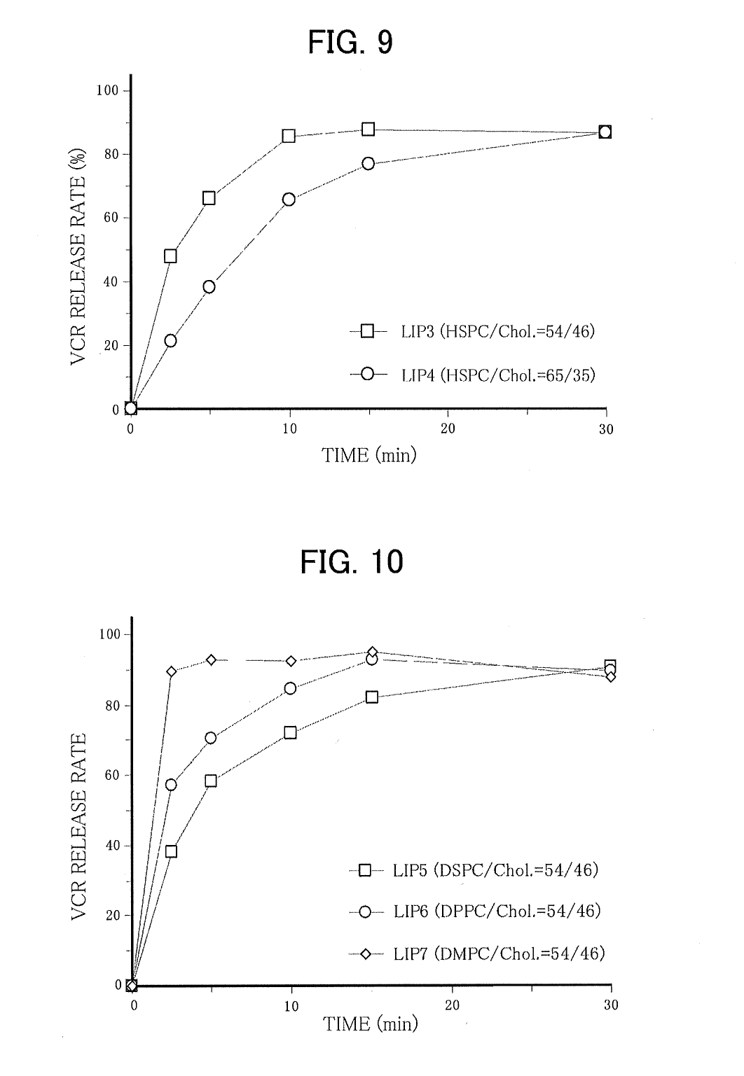

FIG. 9 shows drug releasability from the liposomes having different compositional ratios between the phospholipid and cholesterol.

As to the drug releasability from the liposomes, a significant difference of the drug releasability has been confirmed between the ratios of the phospholipid and the cholesterol of 54:46 and 60:40. Since it is known that the membrane fluidity is changed depending on the content of cholesterol, the above results are considered to be ascribed to the changes of membrane fluidity and drug releasability. Accordingly, the release difference obtained in this example is greatly influenced by the fluidity of liposomes, revealing that the membrane fluidity can be evaluated by the drug releasability evaluation method of the invention.

FIG. 10 shows drug releasability from liposomes made of phospholipids having different phase transition temperatures.

It has been made clear that the VCR release increases in the order of DMPC>DPPC>DSPC. On the other hand, the membrane fluidity indicated in terms of a lipid phase transition temperature becomes higher in the order of DMPC>DPPC>DSPC. It has become evident that with respect to the drug release from liposomes, higher membrane fluidity results in higher releasability. Accordingly, the relation between the drug releasability from liposome and the phase transition temperature obtained in this example are coincident with the past knowledge.

The drug release from liposomes takes part in the structure of a liposome membrane, especially, fluidity, in a pharmacokinetic experiment using animal and an in vitro release experiment using living body components. The drug releasability increases with increasing fluidity of the liposome membrane. This is because when a drug is released from a liposome membrane, the liposome membrane acts as a diffusion barrier. Accordingly, liposome membrane physical properties are very important, additionally to the particle size of liposomes, in the preparation quality control. From these standpoints, it becomes necessary to establish a method of evaluating membrane physical properties in liposome preparations and a testing method of drug release from liposome preparations. In this example, using preparations having different ratios of the phospholipid and cholesterol and preparations having different phospholipid phase transition temperatures, the drug releasability was studied. As a result, it has become evident that these preparations exhibit different release behaviors, making it possible to verify the influence of the membrane fluidity by evaluation of the drug releasability. Moreover, with the case where the membrane fluidity is examined at high sensitivity, there is an indirect evaluation method wherein additives such as a probe, etc., are contained in a liposome membrane and the membrane fluidity is evaluated from the behavior of the probe. In this connection, however, the releasability exhibited in this example does not rely on a probe and the fluidity can be evaluated from the drug releasability.

### Preparatory Example 4

### Preparation of DXR-Containing Liposomes and VCR-Containing Liposomes

### 1. Preparation of DXR-Containing Liposomes and VCR-Containing Liposomes

VCR-containing liposomes and DXR-containing liposomes were prepared according to the following steps.

### (1) Preparation of a Lipid Dispersion

0.71 g of HSPC and 0.29 g of Chol. were, respectively, weighed. These were mixed with 1 ml of absolute ethanol, followed by heating and dissolution in a thermostatic oven at 70° C. to obtain an ethanol solution of the lipids. A 250 mM citric acid aqueous solution and sucrose were added to the ethanol solution to make the osmotic pressure of the ethanol solution at 500 mOsm. Next, 9 ml of a liquid whose pH was adjusted to 2.5 was added to the ethanol solution, followed by further heating to obtain a lipid dispersion.

### (2) Size-Control of Liposomes

The thus obtained lipid dispersion was subjected to size-control by passing through a two-stacked filter (0.1 μm, polycarbonate membrane, made by Whatman Co., Ltd.) attached to an Extruder T.10 (Lipex Biomembranes, Ltd.) heated to about 70° C., thereby obtaining a suspension of liposomes after the size-control.

### (3) Surface Modification of the Liposomes

A PEG5000-DSPE aqueous solution (concentration: about 0.04 g/ml) was provided in such an amount that a content of the PEG5000-DSPE corresponded to 0.75 mol % of the afore-weighed total lipid amount (a sum of HSPC and Chol.). This PEG5000-DSPE aqueous solution was heated in a thermostatic oven preliminarily set at 65° C. The PEG5000-DSPE aqueous solution and the suspension of the liposomes after the size-control were mixed together. After the mixing, the mixture was heated in the thermostatic oven set at 65° C. for further 30 minutes to obtain a PEG-modified liposome suspension.

### (4) Replacement of an External Solution

A gel column (Sephalose 4 Fast Flow, made by Amersham Biosciences Co.) wherein the mobile phase was replaced by a 10% sucrose/10 mM histidine solution (pH: 6.5) was provided. Using this gel column, an external solution of the suspension of the PEG-modified liposomes was substituted with the 10% sucrose/10 mM histidine solution (pH: 6.5) to obtain a suspension of the liposomes after the replacement of the external solution. By the replacement of the external solution, a pH gradient was established between the inside and outside of the liposome membrane.

### (5) Entrapping of a Drug

In the preparation of DXR-containing liposomes, a DXR aqueous solution was added to the suspension of the liposomes after the replacement of the external solution in such a way that a ratio by weight of DXR and HSPC (DXR/HSPC) was given 0.14 (w/w). This was heated in a thermostatic oven at 60° C. for 30 minutes to permit DXR entrapping, thereby obtaining a suspension of the liposomes after the DXR entrapping.

In the preparation of VCR-containing liposomes, a VCR aqueous solution was added to the suspension of the liposomes after the replacement of the external solution in such a way that a ratio by weight of VCR and HSPC (VCR/HSPC) was given 0.22 (w/w). This was heated in a thermostatic oven at 60° C. for 30 minutes to permit VCR entrapping, thereby obtaining a suspension of the liposomes after the VCR entrapping.

### (6) Elimination of a Non-Entrapped Drug

A gel column (Sephalose 4 Fast Flow) wherein the mobile phase was replaced by a 10% sucrose/10 mM histidine solution (pH: 6.5) was provided. Using the gel column, the 10% sucrose/10 mM histidine solution (pH: 6.5) was employed as a mobile phase to eliminate the drug left in the external solution of the suspension of the DXR-entrapped liposomes or VCR-entrapped liposomes to obtain a suspension of liposomes after the elimination of the non-entrapped drug.

Finally, the suspension was filtrated with a filter (0.2 μm) to obtain liposomes after the filtration with the filter.

### 2. Liposome Characteristics of DXR-Containing Liposomes and VCR-Containing Liposomes

In Table 5, there are shown the liposome characteristics of the DXR-containing liposomes (hereinafter abbreviated as “LIP8”) and the VCR-containing liposomes (hereinafter abbreviated as “LIP9”), prepared according to the Preparatory Example 4. It will be noted that the measurement of a drug concentration in the liposomes (quantitative determination of DXR or quantitative determination of VCR) and the measurement of the particle size were, respectively, made according to the methods set out hereinbefore.

TABLE 5
Liposome characteristics

| | | Drug | |
|---|---|---|---|
| | | concentration | Particle size |
| | Drug | [mg/m1] | [nm] |
| LIP8 | DXR | 1.29 | 114.6 |
| LIP9 | VCR | 1.37 | 113.9 |

### Example 6

### Evaluation of DXR and VCR Releasability from Liposomes

In this example, LIP8 and LIP9 were used as liposomes.

### 1. Preparation of a Shift Reagent

Ammonium acetate was dissolved in a phosphate buffer solution having a pH of 7.4 in such a way that an ammonium ion concentration was made at 50 mM. Moreover, sodium chloride was added to the solution to adjust the osmotic pressure to 300 mOsm. The resulting solution was used as a shift reagent solution.

### 2. Quantitative Determination of Released DXR and VCR

Liposomes were diluted with the shift reagent solution at 1:10 and heated at 37° C. Sampling was carried out 2.5, 5, 10, 15 and 30 minutes after commencement of the sampling. It will be noted that the samples were stored under ice cooling before use. The released DXR and VCR were quantitatively determined by the method set out hereinbefore, respectively.

### 3. Results

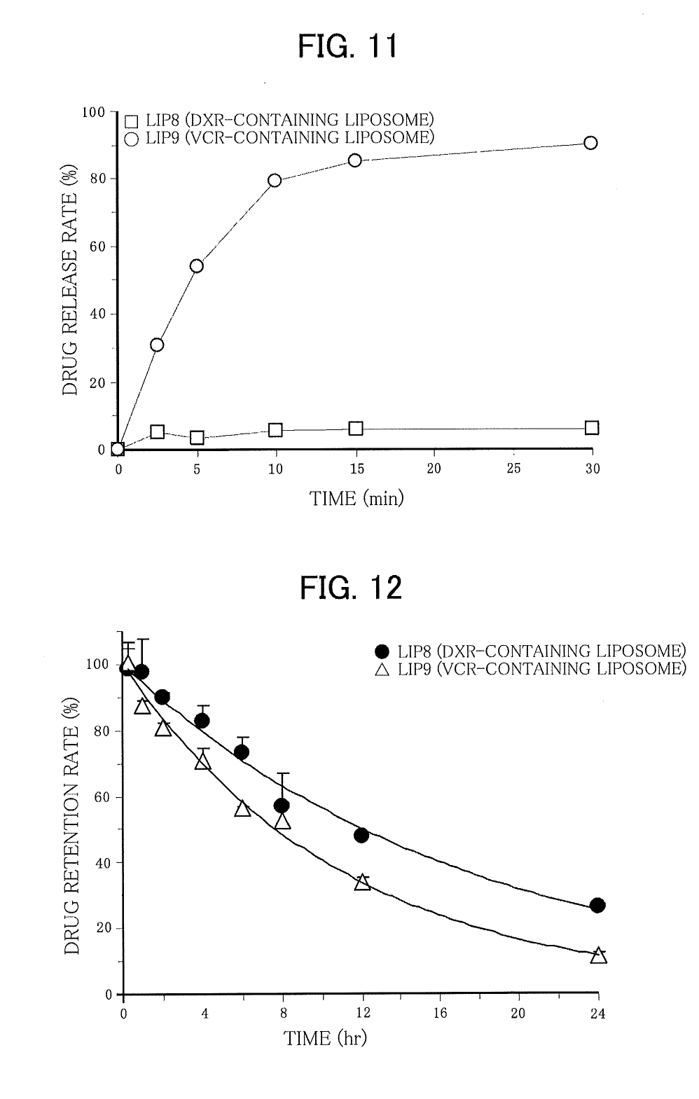

FIG. 11 is a view showing the release behavior of DXR or VCR from the liposomes.

Although the DXR-containing liposomes (LIPS) allowed little drug to be released, the VCR-containing liposomes (LIP9)) were such that the released amount of the drug increased with time.

### Example 7

### Disposition in Blood of DXR-Containing Liposomes and VCR-Containing Liposomes

In this example, the disposition in blood of the DXR-containing liposomes and VCR-containing liposomes was evaluated.

### 1. Material

LIP8 (DXR-containing liposomes) and LIP9 (VCR-containing liposomes) prepared in the Preparatory Example 4 were used as liposomes. As experimental animal for evaluating the disposition in blood, SD rats were used.

### 2. Method

Injection was made from the tail vein of SD rats at a drug dosage of 0.10 μmol/kg.

After the injection, blood sampling from the rat tail vein was carried. The resulting blood was centrifuged (5000 rpm, 10 minutes) to obtain a blood serum. 200 μl of methanol was added to the blood serum and centrifuged (3500 rpm, 10 minutes), followed by collection of a supernatant liquid for use as a sample solution.

The respective sample solutions were subjected to DXR and VCR determination according to the method described hereinbefore. It will be noted that for the DXR determination in this example, a fluorometer was used as a detector wherein the measuring wavelength was set at an excitation wavelength of 485 nm and a fluorescence wavelength of 590 nm and an injection amount was at 100 μl. Also, for the VCR determination in this example, an injection volume was at 50 μl.

### 3. Results

FIG. 12 is a view showing pharmacokinetics of DXR and VCR in blood. Table 6 shows the results of calculating, according to a 1-compartment model, pharmacokinetic parameters from the pharmacokinetics obtained in FIG. 12.

As shown in FIG. 12, the drug disposition of liposomes differs depending on the type of drug. Additionally, as shown in Table 6, it has been revealed that the drug retention is lower for the VCR-containing liposomes (LIP9) than for the DXR-containing liposomes (LIP8).

TABLE 6
Pharmacokinetic parameters

| | Offset half time | |
|---|---|---|
| | (hr) | AUC (% · hr) |
| LIP8 | 12.1 ± 0.1 | 1281 ± 30 |
| LIP9 | 7.7 ± 0.4 | 989 ± 52 |

The elimination half-life of the PEG5000-DSPE-modified liposomes per se relative to rat is about 13 hours (Chem. Pharm. Bull., 51: 336-338, 2003) and half-life of the DXR-containing liposomes is close to that of the liposomes itself. Moreover, because the retentivity of DXR in the DXR-containing liposomes prepared according to the pH gradient method like LIP8 is very high, the disposition of the DXR-containing liposomes in blood are considered to reflect that of the liposomes per se in blood. On the other hand, with respect to the VCR-containing liposomes prepared according to the same pH gradient method, the elimination half-life becomes lower than that of the DXR-containing liposomes, and the VCR is released in the blood. That is, with the DXR-containing liposomes, the drug is retained in the liposomes even in an in vivo environment, and with the VCR-containing liposomes, the drug is released into the external solution of the liposomes in an in vivo environment. These relations can be clarified when using the drug releasability evaluating method of the invention as shown in Example 5.

Accordingly, the method for the drug releasability evaluating method of the invention is one wherein not only a membrane structure change of liposomes and an environmental change of the inner and outer aqueous phases can be evaluated, but also it is a system which enables the evaluation of the releasability from liposomes in blood without doing in vivo testing. Additionally, the method is able to realize the In Vivo/In Vitro correlation that is carried out with respect to oral preparations based on this evaluation method.

### Preparatory Example 5

### Preparation of VCR-Containing Liposomes and CFX-Containing Liposomes

1. Preparation of VCR-Containing liposomes and CFX-Containing Liposomes

VCR-containing liposomes and CFX-containing liposomes were prepared according to the following steps.

### (1) Preparation of a Lipid Dispersion

7.06 g of HSPC and 2.94 g of Chol. were, respectively, weighed. These were mixed with 10 ml of absolute ethanol, followed by heating and dissolution in a thermostatic oven at 70° C. to obtain an ethanol solution of the lipids. Separately, 90 ml of a 250 mM ammonium sulfate aqueous solution was provided and was preliminarily heated in a thermostatic oven at 70° C. This ammonium sulfate aqueous solution was added to the ethanol solution, followed by further heating to obtain a lipid dispersion.

### (2) Size-Control of Liposomes

The thus obtained lipid dispersion was successively passed through filters (polycarbonate membranes, pore sizes 0.2 μm×3 times, 1 μm×10 times, made by Whatman Co., Ltd.) attached to an Extruder T.100 (Lipex Biomembranes, Ltd.) heated to about 70° C., thereby obtaining a suspension of liposomes after the size-control.

### (3) Surface Modification of the Liposomes

A PEG5000-DSPE aqueous solution (concentration: about 37.7 mg/ml) was provided in such an amount that a content of the PEG5000-DSPE corresponded to 0.75 mol % of the afore-weighed total lipid amount (a sum of HSPC and Chol.). This PEG5000-DSPE aqueous solution was heated in a thermostatic oven preliminarily set at 65° C. The PEG5000-DSPE aqueous solution and the suspension of the liposomes after the size-control were mixed together. After the mixing, the mixture was heated in the thermostatic oven set at 65° C. for further 30 minutes to obtain a PEG-modified liposome suspension.

### (4) Replacement of an External Solution

The external solution of the suspension of the PEG-modified liposome was replaced by a 10% sucrose/10 mM histidine solution (pH: 6.5) by use of a cross flow filtration unit (Viva Flow, made by Viva Science CO., Ltd., MWCO 100,000) to obtain a suspension of the liposomes after the replacement of the external solution.

### (5) Entrapping of a Drug

In the preparation of VCR-containing liposomes, a VCR aqueous solution was added to the suspension of the liposomes after the replacement of the external solution in such a way that a ratio by weight of VCR and HSPC (VCR/HSPC) was given 0.10 (w/w) on the basis of the lipid concentration measured by use of a phospholipid determination kit. This was heated in a thermostatic oven at 55° C. for 30 minutes to obtain a suspension of the liposomes after the VCR entrapping.

In the preparation of CFX-containing liposomes, a CFX aqueous solution was added to the suspension of the liposomes after the replacement of the external solution in such a way that a ratio by weight of CFX and HSPC (CFX/HSPC) was given 0.04 (w/w) on the basis of the lipid concentration measured by use of a phospholipid determination kit. This was heated in a thermostatic oven at 55° C. for 30 minutes to obtain a suspension of the liposomes after the CFX entrapping.

### (6) Elimination of a Non-Entrapped Drug

By use of a cross flow filtration unit (Viva Flow 50, made by Viva Science Co., Ltd., MWCO 100,000), while supplying a 10% sucrose/10 mM histidine solution (pH: 6.5) so as to give a constant volume of sample, a non-entrapped drug contained in the liposome suspension after the entrapping of the drug (a liposome suspension after the entrapping VCR or liposome suspension after the entrapping of CFX) was eliminated to obtain a liposome suspension after the elimination of the non-entrapped drug.

Next, the suspension was subjected to quantitative determination of a drug of a liposome after the elimination of the non-entrapped drug.

Finally, the obtained liposome suspension after the elimination of the non-entrapped drug was filtrated with a filter (0.2 μm) to obtain liposomes after the filtration with the filter.

### 2. Liposome Characteristics of VCR-Containing Liposomes and CFX-Containing Liposomes

In Table 7, there are shown the liposome characteristics of the VCR-containing liposomes (hereinafter abbreviated as “LIP10”) and the CFX-containing liposomes (hereinafter abbreviated as “LIP11”), prepared according to the Preparatory Example 5.

TABLE 7
Liposome characteristics

| | | Drug concentration | Particle size* |
|---|---|---|---|
| | Type of Drug | [μmol/ml] | [nm] |
| LIP10 | VCR | 1.04 | 105.5 |
| LIP11 | CFX | 1.02 | 105.5 |

*Indicated as liposomes after the replacement of the external solution.

### Example 8

### Releasability from Liposomes Bearing Different Types of Drugs to an External Solution of the Liposomes

In this example, liposomes used were those of LIP10 and LIP11.

### 1. Preparation of a Shift Reagent Solution

1.9 g of ammonium acetate was weighed, to which 210 ml of 0.2 mols/l of a hydrogen disodium phosphate solution and 40 ml of 0.2 mol/l of a dihydrogen sodium phosphate solution were added, followed by further addition of 500 ml of water to prepare a shift reagent solution having a pH of 6.5.

### 2. Quantitative Determination of Released VCR and CFX

The liposomes prepared in the Preparatory Example 1 were diluted with the shift reagent solution at 1:10 and heated at 37° C. Samples were taken out 5, 10, 15 and 30 minutes after commencement of the heating. It will be noted that the samples were stored under ice cooling before use. The released VCR and CFX were quantitatively determined by the method set out hereinbefore.

### 3. Results

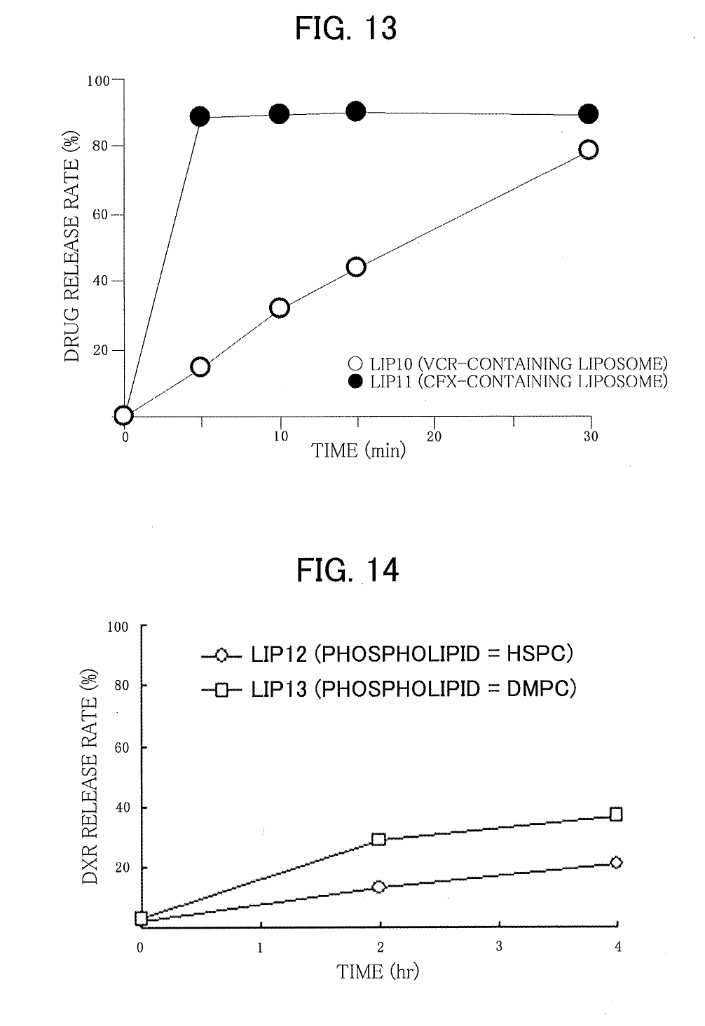

In FIG. 13, there is shown a change of the drug release rate of the VCR-containing liposomes (LIP10) and the CFX-containing liposomes (LIP11) with time.

As will be seen from FIG. 13, the releasability greatly differs depending on the type of entrapped drug even when using the same membrane. This is considered to result from affinity for the membrane.

Accordingly, it will be apparent that the drug releasability evaluating method using the shift reagent shown in this example is a method of evaluating drug affinity for membrane.

### Preparatory Example 6

### Preparation of DXR-Containing Liposomes having Different Types of Phospholipids

### 1. Preparation of DXR-Containing Liposomes

DXR-containing liposomes whose phospholipid was made of HSPC or DMPC were prepared according to the following steps.

### (1) Preparation of a Lipid Dispersion

0.70 g of HSPC and 0.29 g of Chol. were, respectively, weighed. Moreover, 0.67 g of DMPC and 0.33 g of Chol. were, respectively, weighed. These were mixed with 1 ml of absolute ethanol, followed by heating and dissolution in a thermostatic oven at 70° C. to obtain an ethanol solution of the lipids. Separately, 9 ml of a 250 mM ammonium sulfate aqueous solution was provided and was preliminarily heated in a thermostatic oven at 70° C. This ammonium sulfate aqueous solution was added to the ethanol solution, followed by further heating to obtain a lipid dispersion.

### (2) Size-Control of Liposomes

The thus obtained lipid dispersion was successively passed through filters (polycarbonate membranes, pore sizes 0.2 μm×3 times, 0.1 μm×10 times, made by Whatman Co., Ltd.) attached to an Extruder T.10 (Lipex Biomembranes, Ltd.) heated to about 70° C., thereby obtaining a suspension of liposomes after the size-control.

### (3) Surface Modification of the Liposomes

A PEG5000-DSPE aqueous solution (concentration: about 37.7 mg/ml) was provided in such an amount that a content of the PEG5000-DSPE corresponded to 0.75 mol % of the afore-weighed total lipid amount (a sum of HSPC and Chol.). This PEG5000-DSPE aqueous solution was heated in a thermostatic oven preliminarily set at 65° C. The PEG5000-DSPE aqueous solution and the suspension of the liposomes after the size-control were mixed together. After the mixing, the mixture was heated in the thermostatic oven set at 65° C. for further 30 minutes to obtain a PEG-modified liposome suspension.

### (4) Replacement of an External Solution

A gel column (Sephalose 4 Fast Flow, made by Amersham Biosciences Co.) wherein the mobile phase was replaced by a 10% sucrose/10 mM histidine solution (pH: 7.4) was provided. Using this gel column, an external solution of the suspension of the PEG-modified liposomes was substituted with the 10% sucrose/10 mM histidine solution (pH: 7.4) to obtain a suspension of the liposomes after the replacement of the external solution.

### (5) Entrapping of a Drug

A DXR solution was added to the suspension of the liposomes after the replacement of the external solution in such a way that a molar ratio of DXR and the lipid (DXR/total lipids) was made at 0.10 (mol/mol) on the basis of the total lipid concentration measured by use of a phospholipid determination kit. This was heated in a thermostatic oven at 60° C. for 60 minutes to obtain a suspension of the liposomes after the drug entrapping.

### (6) Elimination of a Non-Entrapped Drug

A gel column (Sephalose 4 Fast Flow) wherein the mobile phase was replaced by a 10% sucrose/10 mM histidine solution (pH: 6.5) was provided. Using this gel column and the 10% sucrose/10 mM histidine solution (pH: 6.5) as the mobile phase, the drug left in the external solution of the suspension of the liposomes after the drug entrapping was eliminated to obtain a suspension of the liposomes after elimination of the non-entrapped drug.

Finally, the suspension was filtrated with a filter (0.2 μm) to obtain liposomes after the filtration with the filter.

### 2. Liposome Characteristics of DXR-Containing Liposomes

In Table 8, there are shown the liposome characteristics of the DXR-containing liposomes (LIP12 and LIP13) prepared according to the Preparatory Example 6. The DXR determination, lipid concentration measurement and particle size were, respectively, made according to the methods set out hereinbefore with respect to the liposomes after the filtration with filter.

TABLE 8
Liposome characteristics

| | | Lipid | Drug | | |
|---|---|---|---|---|---|
| | | membrane | concen- | Total lipid | Particle |
| | | composition | tration | concentration | size |
| | Phospholipid | PC²/Chol. | [mg/ml] | [mg/ml] | [nm] |
| LIP12 | HSPC | 54/46 | 1.74 | 11.2 | 107 |
| LIP13 | DMPC | 54/46 | 2.08 | 11.7 | 104.4 |

PC²: Phospholipid (HSPC or DMPC)

### Example 9

### DXR Release Behavior when Using Ethylenediamine as a Shift Reagent

In this example, there is illustrated a DXR release behavior when using ethylenediamine as a shift reagent. It will be noted that LIP12 and LIP13 were used as liposomes.

### 1. Preparation of a Shift Reagent Solution

The shift reagent was weighed, to which a phosphate buffer solution was added, thereby preparing a 250 mM shift reagent solution having a pH of 7.4.

### 2. Quantitative Determination of Released DXR

DXR-containing liposomes (LIP12 and LIP13 prepared in the Preparatory Example 6) were diluted with the shift reagent at 1:10 and heated at 37° C. Samples were taken out 0, 2 and 4 hours after commencement of the heating. It will be noted that the samples were stored under ice cooling before use. Released DXR was quantitatively determined according to the “method of quantitative determination of released DXR” set out hereinbefore.

Where the releasability of DXR from the DXR-containing liposomes by use of the shift reagent solution indicated in Example 8 was evaluated, it had been already stated in Example 6 that little DXR was released.

On the other hand, although the 250 mM ethylenediamine/phosphate buffer solution (pH: 7.4) was used as a shift reagent solution in Example 9, it was revealed that DXR could be released as shown in FIG. 14.

In Example 9, the liposomes prepared by use of different types of phospholipids as a lipid membrane were compared with respect to the DXR releasability. A significant difference in the DXR releasability was recognized between LIP12 using HSPC as a phospholipid and LIP13 using DMPC. The present inventors guessed that this difference was based on a drug releasability change ascribed to the difference in membrane fluidity.

Accordingly, the method of evaluating drug releasability depending on the type of shift reagent shown in Example 9 enables release characteristics to be evaluated with respect to liposomes unlikely to release a drug by proper choice in type of a drug or by choice of a shift reagent based on liposome characteristics.

It is suggested that the characteristic change obtained by the evaluation method shown in Example 9 leads not only to the evaluation of drug release from liposomes, but also to an evaluation method wherein minute physical and chemical changes of the liposomes, which could not be detected by existing measuring devices, can be detected.

### INDUSTRIAL APPLICABILITY

The drug release testing method, etc., of the invention can be used for quality control of evaluating whether drug release characteristics of liposome preparations are within given ranges.

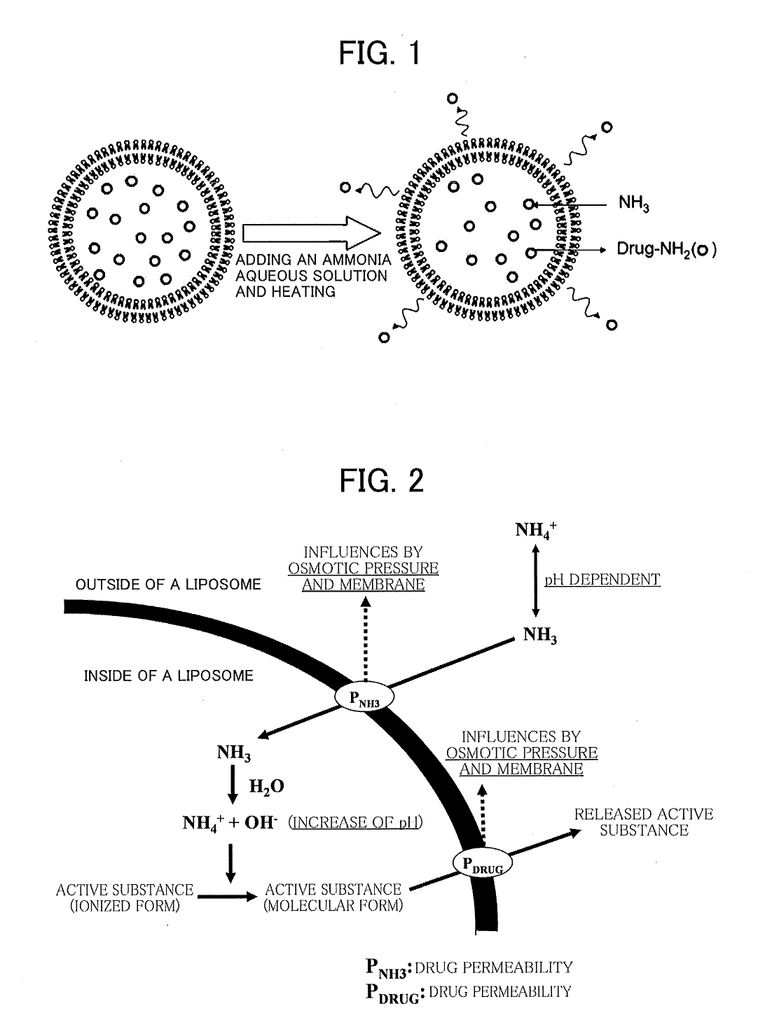

In the drawings:
[FIG. 1]
1-1: Adding and heating an ammonia aqueous solution

### [FIG. 2]

2-1: Outside of a liposome
2-2: Influences by osmotic pressure and membrane
2-3: pH dependent
2-4: Inside of a liposome
2-5: Influences by osmotic pressure and membrane
2-6: Increase of pH
2-7: Released active substance
2-8: Active substance (ionized form)
2-9: Active substance (molecular form)
P_{NH3}: Drug permeability
P_{DRUG}: Drug permeability

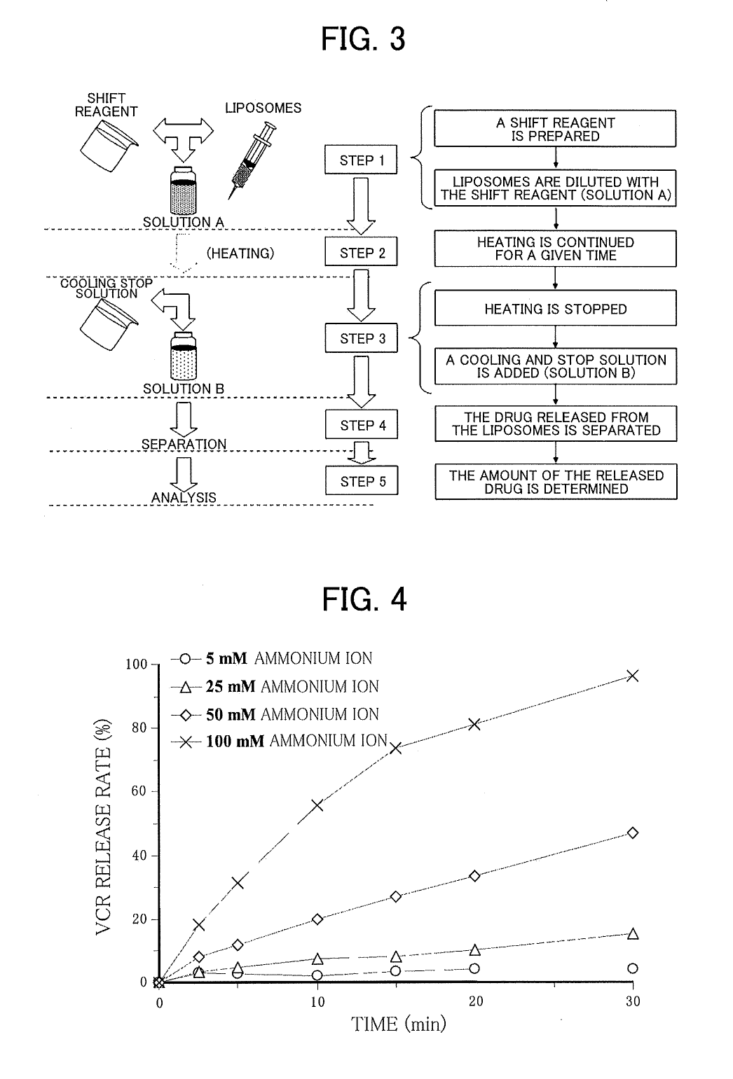

### [FIG. 3]

3-1: Shift reagent
3-2: Liposomes
3-3: Solution A
3-4: Step 1
3-5: A shift reagent is prepared
3-6: Liposomes are diluted with the shift reagent (solution A)
3-7: Heating
3-8: Step 2
3-9: Heating is continued for a given time
3-10: Cooling stop solution
3-11: Solution B
3-12: Step 3
3-13: Heating is stopped
3-14: A cooling and stop solution is added (solution B)
3-15: Separation
3-16: Step 4
3-17: The drug released from the liposomes is separated
3-18: Analysis
3-19: Step 5
3-20: The amount of the released drug is determined

### [FIG. 4]

4-1: Ammonium ion
4-2: VCR release rate (%)
4-3: Time (min)

### [FIG. 5]

5-1: Release rate constant (mg/ml/min)
5-2: Concentration of shift reagent (mM)

### [FIG. 6]

6-1: VCR release rate (%)
6-2: Time (min)

### [FIG. 7]

7-1: VCR release rate (%)
7-2: Time (min)

### [FIG. 8]

8-1: Ammonium acetate
8-2: Diethylamine
8-3: 2-aminoethanol
8-4: Ethylenediamine
8-5: VCR release rate (%)
8-6: Time (min)

### [FIG. 9]

9-1: VCR release rate (%)
9-2: Time (min)

### [FIG. 10]

10-1: VCR release rate (%)
10-2: Time (min)

### [FIG. 11]

11-1: LIP8 (DXR-containing liposome)
11-2: LIP9 (VCR-containing liposome)
11-3: Drug release rate (%)
11-4: Time (min)

### [FIG. 12]

12-1: LIP8 (DXR-containing liposome)
12-2: LIP9 (VCR-containing liposome)
12-3: Drug retention rate (%)
12-4: Time (hr)

### [FIG. 13]

13-1: LIP10 (VCR-containing liposome)
13-2: LIP11 (CFX-containing liposome)
13-3: Drug release rate (%)
13-4: Time (min)

### [FIG. 14]

14-1: LIP12 (Phospholipid=HSPC)
14-2: LIP13 (Phospholipid=DMPC)
14-3: DXR release rate (%)
14-4: Time (hr)

1. A method for evaluating drug releasability of a liposome preparation, wherein liposomes entrapping a drug therein are permitted to be present in a solution, to which a shift reagent has been added, and a concentration of said drug in said solution is measured.

2. The method as defined in claim 1, wherein a chemical equilibrium is caused to be shifted in an inner aqueous phase of said liposomes, so that said drug is released to an outer aqueous phase of said liposomes.

3. The method as defined in claim 1, wherein the liposomes entrapping said drug therein are made of liposomes entrapping the drug according to a remote loading method.

4. The method as defined in claim 1, wherein said shift reagent permeates a lipid membrane of said liposomes in a non-ionized state, moves from the outer aqueous phase to the inner aqueous phase and is cationized to non-ionize the drug retained in the inner aqueous phase.

5. The method as defined in claim 1, wherein said solution is made of a buffer solution.

6. A method for evaluating drug releasability of a liposome preparation comprising the following steps of:
(1) preparing a solution, to which a shift reagent has been added;
(2) mixing liposomes entrapping a drug therein with said solution;
(3) starting release of said drug into said solution;
(4) separating said liposomes from said solution; and
(5) measuring a concentration of the drug released from said solution.

7. The method as defined in claim 6, wherein in the step (3), the solution containing said liposomes is heated for a given time at a given temperature.

8. The method as defined in claim 6, further comprising the following step between the steps (3) and (4):
(3-2) stopping the release of said drug into said solution.

9. The method as defined in claim 8, wherein in the step (3-2), a stop solution is added to the solution containing said liposomes.

10. The method as defined in claim 6, wherein said solution is made of a buffer solution.

11. The method as defined in any of claims 1 to 10, wherein said drug is made of an amphiphatic compound.

12. The method as defined in any of claims 1 to 10, wherein said shift reagent is at least one selected from the group consisting of ammonia and an amino compound having a molecular weight of not larger than 500.
